(19) United States
(12) Patent Application Publication
(10) Pub. No.: US 2008/0124308 A1
(43) Pub. Date: May 29, 2008

(54) Gene Therapy of Solid Tumours by Means of Retroviral Vectors Pseudotyped With Arenavirus Glycoproteins

(76) Inventors: M. Dorothee Laer, Frankfurt (DE); Hrvoje Miletic, Bergen (NO); Yvonne Fischer, Malmo (SE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 11/632,302

(22) PCT Filed: Jul. 14, 2005
(86) PCT No.: PCT/EP05/07687

§ 371 Date: May 9, 2007

(30) Foreign Application Priority Data
Jul. 16, 2004 (DE) 10 2004 034 461

(51) Int. Cl. A61K 35/00; A61P 43/00

(52) U.S. Cl. 424/93.21; 424/93.2

(57) ABSTRACT

The present invention concerns the use of packaging cells which produce retroviral virions pseudotyped with arenavirus glycoprotein, for the manufacture of a pharmaceutical composition for the gene therapy of solid tumors. The invention further concerns the use of the virions which are produced by these packaging cells, for the manufacture of a pharmaceutical composition for the gene therapy of solid tumors. Furthermore, packaging cells which are suitable for this application and pharmaceutical compositions containing these cells are subject matter of the invention.

The present invention concerns the use of packaging cells which produce retroviral virions pseudo-typed with arenavirus glycoprotein, for the manufacture of a pharmaceutical composition for the gene therapy of solid tumors. The invention further concerns the use of the virions which are produced by these packaging cells, for the manufacture of a pharmaceutical composition for the gene therapy of solid tumors. Furthermore, packaging cells which are suitable for this application and pharmaceutical compositions containing these cells are subject-matter of the invention.

Retroviral vectors are increasingly used in the art, for example, for the gene transfer in the genetic or medical research or in gene therapeutic approaches (c.f., e.g., C. Baum et al. in Seminars in Oncology: Gene Therapy of Cancer: Translational approaches from preclinical studies to clinical implementations, eds. Gerson & Lattime, Academic Press, 1998). The retroviral vectors are mostly derived from murine leukemia viruses (MLV) and contain all sequences of the LTR regions which are necessary for the integration and the Ψ-element responsible for the packaging. The regions coding for the virus proteins are replaced by foreign genes and control sequences which are to be introduced into human cells. The vectors are produced in the so-called helper cell lines (packaging cell lines) which generally contain a copy of the coding regions of a complete retroviral genome. It synthesizes all proteins necessary for the replication and infection, but cannot package its genomic virus RNA in particles, as it contains a defect in the Ψ-sequences. When the retroviral vectors are introduced and transcribed in these helper cells, the produced transgene mRNA can interact with the structural proteins of the helper virus through its own Ψ-region and be packaged into particles. The recombinant virions which contain no genetic information for virus components, absorb on cells through their surface proteins; the capsides are taken up into the cytoplasm; and the transgene RNA is transcribed into double-stranded DNA and is integrated into the host cell genome. The advantage of this system is the stable integration of the foreign genes which are passed to the daughter cells during cell division. Of disadvantage is the retroviral own non-specific integration at random positions of the cell genome.

Retroviral vectors provide for a stable, collinear integration (i.e. without recombination and rearrangement of the coding sequences in the vector genome) and, hence, for a long-term expression of the transgene. Up to now long-term gene expression is otherwise only possible with the episomal Herpes virus vectors or the adeno-associated virus vectors (AAV-vectors). For the latter vector systems, the packaging systems (packaging cell lines) are, however, not yet optimized. Moreover, AAV-vectors possess a low packaging capacity (app. 5 kb for AAV as opposite to app. 10-12 kb for retroviral vectors).

In packaging cells, the vector genome which contains retroviral cis-elements is built through transcription. This genomic vector transcript codes for the gene to be transferred, but not for retroviral proteins. However, it will be inserted in an infectious but non-replicative virion in the packaging cell lines with the help of the gag, pol and env gene products from the packaging cell. This virion can then be inserted into the desired cell line as a retroviral vector for the transfer of the transgene integrated into the vector genome, wherein the vector does not further multiply. In other words, the viral vector can infect the target cells, but cannot further multiply therein.

The development of retroviral packaging systems is already far advanced; and vector elements which are free from replication competent viruses can be produced in large amounts under GMP conditions (good manufacturing practice; Commission Directive by the 91/356/EEC of 13 Jun. 1991 laying down the principles and guidelines of good manufacturing practice for medicinal products for human use). Vectors based on the murine leukemia virus (MLV vectors) were already repeatedly used in clinical studies (P. Chu et al., J. Mol. Med. 76 (1998) 184-192).

Two basic types of retroviral packaging systems are known in the art (J. M. Wilson, Clin. Exp. Immunol 107 Suppl. 1 (1997) 31-32; C. Baum et al. (1998), loc. cit.).

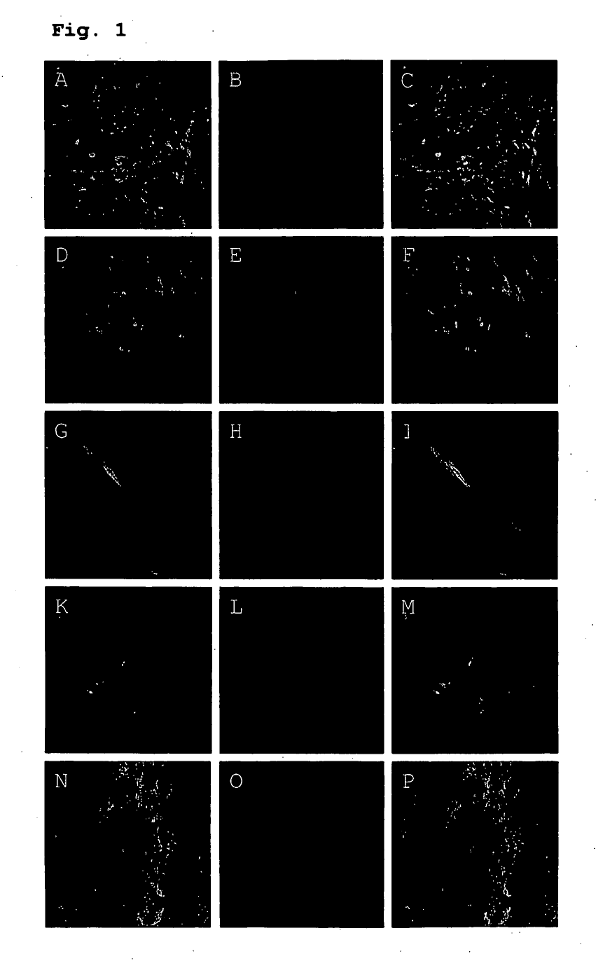

On the one hand, oncoretroviral packaging systems are used. MLV packaging cell lines contain the retroviral genes gag, pol and env (FIG. 1); and the sequences which are necessary for the packaging of the retroviral RNA are deleted (C. Baum et al. (1998), loc. cit.).

The second type of the known packaging systems is derived from the lentiviruses (R. Carroll et al., J. Virol. 68 (1994) 6047-6051; P. Corbeau et al., Proc. Natl. Acad. Sci. USA 93 (1996) 14070-14075; L. Naldini et al., Science 272 (1996) 263-267; C. Parolin et al., J. Virol. 68 (1994) 3888-3895; J. Reiser et al., Proc. Natl. Acad. Sci. USA 93 (1996) 15266-15271; J. H. Riochardson et al., J. Gen. Virol. 76 (1995) 691-696; T. Shimada et al., J. Clin. Invest. 88 (1991) 1043-1047). Lentiviruses are complex retroviruses which express a number of regulatory genes in addition to the gag, pol and env gene products. Examples of lentiviruses from which packaging systems are derived are the human immunodeficiency virus (HIV), the simian immunodeficiency virus (SIV), the equine infectious anaemia virus (EIAV) and the feline immunodeficiency virus (FIV). The structure of the lentiviral packaging systems is in principle similar to that of the MLV vectors.

The advantage of the lentiviral vectors is that they can also infect resting cells. With MLV vectors, however, the vector genome can only be transported into the cell nucleus during the cell division, i.e., when the nuclear membrane is dissolved. However, packaging systems derived from lentiviruses have disadvantages such as relatively low titer and low safety which result from the complex structure of the lentiviral genome. The complex genomic structure does not allow to clearly separate cis- and trans-elements in the genome. Therefore, important cis-regulatory sequences (e.g., parts of the packaging signal) which must also be contained in the vector genome are also provided in the packaging constructs which express the lentiviral gag, pol and env genes. These homologies can lead to recombination between vector genomes and the packaging constructs and, hence, to release of replication competent retroviruses (e.g., an HIV wild-type virus, which would be highly undesirable), so that these systems are not comparable with MLV packaging cell lines from the safety standpoint. However, not all lentiviruses are infectious for humans or the species to be treated, so that the safety of the system can be increased by the selection of a suitable retrovirus which is not infectious for the species, as recombination is unlikely in this case.

To solve the problem that retroviral vectors can in most instances only be produced in insufficient titers and cannot be concentrated or purified without loss of infectiousness due to the instability of their envelope proteins, the vectors can be pseudotyped with the rhabdoviral G protein of vesicular choriomeningitis virus (VSV) (Emi et al. J. Virol. 65 (1991) 1202-1207; J. C. Burns et al., PNAS 90 (1993) 8033-8037; T. Friedmann et al., Nat. Med. 1 (1995) 275-277; D. von Laer et al., J. Virol. 72 (1998) 1424-1430); R. A. Weiss (1993), In: J. A. Levy (ed.), The Retroviridae, Plenum Press, New York).

These kinds of retroviral vectors which are pseudotyped with VSV G protein were already adopted in the art for the therapy of solid tumors. The use of these vectors for the gene transfer of herpes simplex virus thymidine kinase (HSV-tk) with subsequent ganciclovir treatment in a rat glioma model showed a high efficiency of the transduction and a good therapeutic effect (Galipeau et al., Cancer Res. 59 (1999) 2384-2394). The main problem with the pseudotyping with VSV G is, however, the toxicity of the VSV G protein.

In addition, the administration of virions alone is often not sufficient for the effective therapy of a solid tumor to provide a sufficient transduction of all tumor cells. Factors which play a role in this regard are primarily the size of the tumor as well as its vascularisation. Therefore, attempts were already made in the art to administer the packaging cells producing virions instead of the virions themselves. For this purpose, classical packaging cell lines such as fibroblasts which release amphotropic MLV vectors were used. These attempts resulted in an improved but still not sufficient therapeutically effective transduction of the tumor tissue (e.g., Shand N, Weber F, Mariani L, Bernstein M, Gianella-Borradori A, Long Z, Sorensen A G, Barbier N., Hum Gene Ther. 199 September 20; 10(14): 2325-35. A phase 1-2 clinical trial of gene therapy for recurrent glioblastoma multiforme by tumor transduction with the herpes simplex thymidine kinase gene followed by ganciclovir, GLI328 European-Canadian study group). Altogether the failing clinical success of the gene therapy of gliomas with retroviral vectors is attributed not to the ineffectiveness of the therapeutic genes, but to the lack of gene transfer efficiency.

Therefore, the object for the skilled person is to provide suitable systems for the preparation of a gene therapeutic pharmaceutical composition, particularly for the gene therapy of solid tumors, which avoid the disadvantages of the common prior art systems and are capable of effective but nonetheless targeted transduction of tumor cells.

This object is realized in the subject-matter of the following patent claims. In particular, the invention concerns packaging cells which produce retroviral virions which are pseudotyped with arenavirus glycoprotein and which are able to infiltrate solid tumors, as well as the use of these packaging cells for the manufacture of a pharmaceutical composition for the gene therapy of solid tumors. Further provided is a pharmaceutical composition which comprises the packaging cells of the invention.

It could furthermore be determined that packaging cell lines which were previously used for the gene therapy, do not penetrate into solid tumors, but remain in the tumor periphery. In contrast thereto, the inventors surprisingly found that some primary cells (i.e., not immortalized cells obtained ex vivo or after culturing which does not essentially contribute to the cell differentiation) or cell lines, in particular stem cells, which are derived therefrom by culturing under certain conditions, are able to infiltrate solid tumors.

The capability to infiltrate solid tumors is reflected in the fact that at least 50%, preferably at least 60%, most preferably at least 70% or at least 80% of the detected cells reside within the tumor after app. 1 to 5 days, preferably also up to 20 days, after injection of the cells in a solid tumor or in its direct vicinity. Thereby, preferably at least 50%, most preferably at least 75% of the tumor mass is infiltrated. A suitable test is described in detail in the Examples.

Stem cells possess particularly positive migration properties. According to the invention, stem cells comprise multipotent as well as pluripotent stem cells. These cells can be produced according to known methods. It is, however, preferred that adult stem cells are used as packaging cells to avoid ethical problems relating to the use of embryonic stem cells. Among the stem cells which have already undergone a basic differentiation are, for example, mesenchymal stem cells (MSC). Experiments show that MSC of different origin possess the ability to infiltrate solid tumors, in particular brain tumors. A particular ability for this purpose was shown also for multipotent adult progenitor cells (MAPC). The generation of this kind of cells is known in the art (e.g., Y. Jiang et al., Nature 418 (1992)41-49). Therefore the use of MSC and/or MAPC as packaging cells for the manufacture of a pharmaceutical composition for the treatment of solid tumors is a preferred embodiment of the invention.

It is further known in the art that, e.g., T lymphocytes which carry a T-cell receptor which specifically recognizes a tumor antigen, can infiltrate solid tumors. Tumor infiltrating lymphocytes can, e.g., be isolated from a tumor after the resection. An attempt was already made to employ the migration properties of these cells in therapy by introducing, e.g., the immunostimulating gene IL-2 into tumor infiltrating lymphocytes (TIL). This kind of transgenic TIL can in some cases lead to tumor regression (S. A. Rosenberg et al., N. Engl. J. of Med. 319 (1988) 1676-1680). Also tumor infiltrating lymphocytes can be used as packaging cells for the purpose of this invention.

To minimize xenogenic immune reactions against the packaging cells, those packaging cells which are derived from the species to be treated are preferably used for the gene therapy. Preferably the packaging cells are of human origin. It is possible to use autologous cells as packaging cells. As the cells only shortly remain in the tumor, one can as well take allogenic cells. An allogenic cell would be preferable for safety reasons (development of an allogenic, neoplasia from the packaging cells is unlikely) as well as for the reasons of simplified manufacture (ready medicament vs. individual formulation). Some inflammation is even beneficial.

A precondition which enables gene therapy of tumors with viral vectors is the specificity of the transduction of the tumor cells by the vectors. An unspecific transduction of non-tumor cells leads to side effects within the scope of the gene therapy, as the gene therapy in general aims at the destruction of the transduced cells. The infiltration of the packaging cells of the invention into the tumor therefore reduces the probability that the virions produced by the packaging cells transduce cells outside the tumor.

An increase in specificity and a decrease in side effects can further be achieved by the fact that the therapeutically applicable genes transferred by the retroviral vector are placed under the expression control of a promoter which is specifically activated in tumor cells, as opposed to the use of constitutive promoters. In this case, however, it should be tested whether the tumor-specific promoter indeed leads to an expression in the tumor cells, whereas neighboring cells do not activate this promoter.

A further crucial factor for the specificity of the transduction is, however, the tropism of the virions. It is determined essentially by the envelope protein of the virus.

In previous experiments, a very broad spectrum of target cells for retroviral vectors pseudotyped with LCMV glycoprotein was found (W. Beyer et al., J. Virol 76 (2002)1488-1495). Fibroblast cell lines, epithelial cell lines, glioma and neuroblastoma cell lines, myeloid progenitor cell lines, a hepatoma cell line and thymus cell line from the species human, hamster, dog and mouse can be transduced with high efficiency by the vectors pseudotyped with LCMV glycoprotein. Also primary glioblastoma cells and oligodendroglioma cells could be successfully transduced. Various authors have already investigated the in vivo transduction pattern of retroviral vectors in the brain which were pseudotyped with different glycoproteins, among others with LCMV glycoprotein. Transduction of different cells of the striatum, thalamus and corpus callosum was found, albeit with lower efficiency than with other vectors, e.g., vectors pseudotyped with VSV G protein (D. Watson et al., Mol. Ther. 5 (2002) 528-537; L.-F. Wong et al., Mol. Ther. 9 (2004) 101-111).

It could now be surprisingly found that retroviral vectors pseudotyped with arenavirus glycoprotein, in particular with LCMV glycoprotein, specifically transduce brain tumors in vivo. The subject-matter of the invention is therefore the use of retroviral virions pseudotyped with arenavirus glycoprotein for the manufacture of a pharmaceutical composition for the gene therapy of solid tumors, particularly brain tumors. The use of virions is particularly advisable in small or well vascularized tumors, as in this case the amount of virions delivered with one or a few applications can be sufficient for the transduction of an adequate amount of tumor cells, although as complete as possible transduction of all tumor cells is preferred. To limit the number of applications, it is advisable, in particular in large tumors, to use packaging cells which produce retroviral virions pseudotyped with arenavirus glycoprotein (in particular LCMV glycoprotein) for the manufacture of the pharmaceutical composition for the treatment of solid tumors. The subject-matter of the invention is therefore also the use of packaging cells which produce retroviral virions pseudotyped with arenavirus glycoprotein (in particular, LCMV glycoprotein) for the manufacture of a pharmaceutical composition for the gene therapy of brain tumors. The use of packaging cells which are able to infiltrate brain tumors for the manufacture of a pharmaceutical composition for the gene therapy of brain tumors is particularly preferred. The packaging cells or the vector are applied either by stereotactic injection or by introduction into the interior or by injection into the wall of the resection cavity after operative removal of the tumor.

According to the invention, solid tumors are all tumors which are not of haematopoietic origin such as, for example, carcinomas, e.g., mamma carcinoma or sarcoma. The tumor can, for example, be a glioma, neuroblastoma, oligodendroglioma or astrocytoma. The tumor can be benign, although malignant tumors, in particular a malignant glioma, are preferred. Malignant gliomas represent the most common brain tumor and often result in patient's death (Y. Kew et al., Curr. Opin. Neurol. 16 (2003) 665-670). Solid primary tumors as well as individual tumor cells and metastases migrated therefrom are designated as solid tumors according to this invention.

The packaging cells of the invention produce retroviral virions pseudotyped with arenavirus glycoprotein, in particular LCMV glycoprotein.

Experiments have shown that these virions specifically infect the cells of a brain tumor in vivo. In particular, the virions do not infect neurons during the tumor therapy in vivo. According to this invention, this means that less than 5%, preferably less than 2*, almost none or none of the neighboring neurons are infected by injection into a brain tumor. Furthermore the virions infect only a small number of astrocytes, preferably less than 15%, less than 10%, or less than 5% of the neighboring astrocytes during the tumor therapy in vivo. On the other hand, even individual metastasized tumor cells were infected by the virions. This high specificity for the tumor cells makes the virions particularly suitable for the treatment of tumors, mostly solid tumors and, in particular, brain tumors.

According to the present invention, an arenavirus glycoprotein, e.g., of Lassa or LCMV, is employed for the pseudotyping. Thereby it is possible or it can even be preferred that a glycoprotein of another LCMV or Lassa strain is employed instead of the wild-type glycoprotein. In this way slight variations in the gp nucleic acid sequence or in the amino acid sequence of the expressed envelope protein in different strains can significantly change the cell tropism (host cell spectrum) (M. Matloubian et al., J. Virol. 67 (1993) 7340-7349; M. N. Teng, J. Virol. 70 (1996) 8438-8443; King et al., J. Virol 64 (1990) 5611-5616). A targeted transduction of the desired cell type is, hence, enabled according to the invention. According to a preferred embodiment of the invention, it can be of advantage to produce packaging systems with different glycoprotein variants (GP variants) for different applications such as the therapy of different solid tumors. This kind of variants are disclosed, e.g., in M. Matloubian et al., J. Virol 67 (1993) 7340-7349 or M. N. Teng et al., J. Virol. 70 (1996) 9438-8443. The tropism of variants with regard to a particular tumor type can be tested experimentally, as shown, for example, for glioblastomas.

Also, wild-type LCMV can infect different cell types from different tissues and species. It was shown that at least alpha dystroglycan with broad expression can be a receptor for LCMV (P. Borrow et al., J. Virol. 66 (1992) 7270-7281; W. Cao et al., Science 282 (1998) 2079-2081). In the flexibility of the tropism through mutations of the glycoprotein there can be seen an indication that the glycoproteins can bind to different closely related receptors or to a receptor with different post-translational modifications.

The envelope proteins of the arenaviruses are initially expressed as precursor polypeptide, GP-C, which is cleaved post-translationally into GP-1 and GP-2 by a cellular protease. Thereby GP-1 interacts with the alpha dystroglycan receptor, whereas GP-2 contains the peptide responsible for the fusion and the trans-membrane domain.

According to the present invention, one can use the gp genes of the rather neurotropic LCMV strain Armstrong, L (ARM) (L. Villarete et al., J. Virol. 68 (1994) 7490-7496) (for SEQ ID NO: 4 coding region; cf. Annex to the sequence protocol, TO SEQ ID NO: 3). The rather haematotropic strain WE (V. Romanowski et al., Virus Res. 3, (1985) 101-114) (SEQ ID NO: 1) is preferably used. Particularly preferred is the pseudo-typing with a variant of LCMV-WE-HPI (LCMV-WE-HPIopt, see SEQ ID NO: 27), in which the cordon usage was optimized and “cryptic splice regions” were deleted to achieve an improved expression.

The pseudotyping can be improved by any such optimization of the GP expression to dispense with an additional support by at least one further LCMV protein.

A preferred variant is the LCMV glycoprotein WE-HPI described in the present invention. The coding nucleic acid sequence gp (open reading frame (ORF) represented in SEQ ID NO: 25) contains mutations in the positions 281, 329, 385, 397, 463, 521, 543, 631, 793, 1039, 1363 and 1370 compared to the LCMV strain WE and therefore contains amino acid substitutions in the positions 94, 110, 129, 133, 155, 174, 181, 211, 265, 347, 455 and 457 compared to the glycoprotein of the LCMV strain WE. This GP variant with the amino acid sequence shown in SEQ ID NO: 26 has the advantage that it is stable also without additional LCMV helper proteins and effects an improved pseudotyping compared to the strain WE. It was shown that only one of the mutations which the initially disclosed sequence of the LCMV glycoprotein LCMV-WE contains compared to the newly cloned sequence LCMV-WE-HPI, a leucine to proline mutation at amino acid 110, influences the processing of the expressed protein and, hence, the expression on the cell surface (W. Beyer et al., J. Virol. 75 (2001) 1061-1064). Therefore, variants with this mutation should preferably be not used for the pseudotyping.

The invention therefore, also concerns the use of a variant of the lymphocyte choriomeningitis virus which contains the gp gene that codes for the sequence represented in SEQ ID NO: 26 or a part thereof, whereby the gp gene preferably has the sequence represented in SEQ ID NO: 25 or a part thereof. Furthermore, a protein with the amino acid sequence represented in SEQ ID NO: 26 or a part thereof as well as a nucleic acid sequence coding for this protein, preferably the sequence represented in SEQ ID NO: 25 or a part thereof, are part of the invention. These virus variants as well as the latter nucleic and amino acid sequences, e.g., derived from the LCMV variant WE, are obtainable by methods generally known to the skilled person (e.g. by introduction of point mutations).

As already mentioned, “LCMV” according to the invention comprises next to the LCMV wild type also other LCMV strains, in particular LCMV-WE-HPI (see SEQ. ID NO: 25) or the artificially produced variant LCMV-WE-HPIopt (see SEQ ID NO: 27) with optimized codon usage and splice region deletions. Within the scope of the invention, in particular a glycoprotein can be used for the pseudotyping with the nucleotide sequence of the corresponding gene which codes for a glycoprotein with at least 80% homology to the amino acid sequence of the glycoprotein of LCMV wild type or LCMV-WE, LCMV-WE-HPI, LCMV-WE-HPIopt or Lassa virus (see SEQ ID NOs: 1, 25, 27, 28). The homology is preferably at least 90%, at least 95% or app. 99%.

A particularly preferred embodiment of this invention concerns packaging cells for the gene therapy of solid tumors which comprise one or more expression cassettes for the retroviral genes gag, pol and further a gene coding for an arenavirus glycoprotein, whereby the arenavirus glycoprotein is, in particular, Lassa virus glycoprotein.

For the expression of the glycoprotein, expression vectors are generally suitable which allow a high stable gene expression in eukaryotic cells. The choice of the expression vector is, however, only critical as long as it provides for a high and stable expression level, i.e., an expression level which is high enough to allow the generation of pseudotypes, and is long-lasting (stable), without that the promoter switches off.

According to the invention, especially preferred are the following two expression cassettes (S. Mizushima, Nucleic Acids Res. 18 (1990) 5322, T. Uetsuki, J. Biol. Chem. 264 (1989) 5791-5798):

### (CMV promoter)-(β-globin intron-2)-(gp)-(SV40 poly-A signal)

and

### (EF-1alpha promoter)-(gp)-(poly-A signal of G-CSF gene).

The sequences of the elements of the expression cassettes are represented in the sequence protocol or are generally known:
Cytomegalovirus promoter (CMV promoter): (M. Boshart et al., Cell 41 (1958) 521-530; F. Langle-Rouault et al., Virol. 72(7) 6181-5 (1998)).
beta-globin intron-2: (Jeffreys, A. J. et. al., Cell 12 (1977) 1097-1108).
SV40 poly-A signal: (M. Boshart et al., Cell 41 (1958) 521-530; F. Langle-Rouault et al., Virol. 72(7) 6181-5 (1998).
EF-1alpha promoter: SEQ ID NO: 9 (S. Mizushima, Nucleic Acids Res. 18 (1990) 5322, T. Uetsuki, J. Biol. Chem. 264 (1989) 5791-5798).
G-CSF poly-A signal: (S. Mizushima, Nucleic Acids Res. 18 (1990) 5322, T. Uetsuki, J. Biol. Chem. 264 (1989) 5791-5798).
gp (LCMV): cf., e.g., SEQ ID NO: 1, 3, regions coding for SEQ ID NO: 4 (see also Annex to the sequence protocol).
gp (Lassa): cf. SEQ ID NO: 28.

Exemplified sequences for this kind of expression plasmids are deposited in the EMBL data bank under the accession numbers AJ318512 (pHCMV-LCMV-GP(WE), AJ318513 (pHCMV-LCMV-GP(WE-HPI)).

Modifications of the corresponding nucleic acid sequences are possible as long as the functionality of the expression cassette remains unchanged, i.e., its use according to the invention allows for the pseudotyping and the transfection of the target cells; and a stable integration of the transgene in the host genome is not impeded.

Furthermore, an episomal EBV expression vector (Epstein-Barr virus; cf. F. Langle-Rouault et al., Virol. 72(7) (1998) 6181-5) (pCep4) of Invitrogen also shows high expression and is therefore preferred in the context of the present invention.

Retroviruses which are pseudotyped with the arenavirus lymphocytic choriomeningitis virus (LCMV) and retroviral packaging systems suitable therefor are known in the art (EP 1 006 196; Miletic et al., J. Virol. 73 (1999) 6114-6116). The vectors described therein which are pseudotyped with LCMV glycoprotein are suitable for the use in the manufacture of a pharmaceutical composition for the gene therapy of brain tumors. Packaging cells are generally used for the pseudotyping which are deficient in the retroviral envelope protein env, so that virions will be produced only when an envelope protein is otherwise provided, e.g., by infection of the cells with the virus, e.g., LCMV, or by transduction with a plasmid with an expression cassette for the corresponding envelope protein, e.g., the glycoprotein of LCMV.

Compared to vectors which contain the frequently used amphotropic murine leukemia virus envelope protein (A-MLVenv) or VSV G, LCMV-pseudotyped vectors show a similar efficiency in the production and stability. However, in contrast to VSV G, LCMV glycoprotein allows the generation of stable packaging cell lines which constitutively express the glycoprotein, as this protein does not have a cytopathic effect. The pseudotyped virions are also stable enough to be highly concentrated by ultracentrifugation, so that LCMV-pseudotyped retroviral vectors possess basic preconditions for the suitability for the gene therapy.

A retrovirus which is pseudotyped with the arenavirus glycoprotein can be an oncoretrovirus or a lentivirus. A commonly used oncoretrovirus is, e.g., MLV (murine leukemia virus), in particular MoMLV (Moloney MLV). However, it is preferred that one uses a lentivirus, in particular HIV (human immunodeficiency virus), SIV (simian immunodeficiency virus), EIAV (equine infectious anaemia virus) or FIV (feline immunodeficiency virus), as lentiviruses can also transduce resting cells.

The packaging cells comprise the retroviral genes gag (coding region for SEQ ID NO: 12; cf. Annex to the sequence protocol, TO SEQ ID NO: 11), pol (coding region for SEQ ID NO: 13; cf. Annex to the sequence protocol, TO SEQ ID NO: 11) and if necessary the retroviral gene env (coding region for SEQ ID NO: 14; cf. Annex to the sequence protocol, TO SEQ ID NO: 11) and/or regulatory retroviral genes (in case of lentiviral packaging systems, e.g., the gene coding for the lentiviral Rev protein which prevents splicing of the retroviral genomic RNA) and further the gp gene coding for the glycoproteins GP-1 and GP-2 of an arenavirus (LCMV: e.g., coding region for SEQ ID NO: 4; cf. Annex to the sequence protocol, TO SEQ ID NO: 3; Lassa: SEQ ID NO: 28) or part thereof. The indicated sequences refer to MoMLV as an example; the sequences of the genes of other retroviruses are also known in the art. According to a particular embodiment, the packaging systems can also contain the gag and pol gene products of lentiviruses. In this context, it can be necessary that additional accessory lentiviral genes such as rev (coding region for SEQ ID NO: 21; cf. Annex to the sequence protocol, TO SEQ ID NO: 15) or tat (coding region for SEQ ID NO: 20; cf. Annex to the sequence protocol, TO SEQ ID NO: 15) are expressed with HIV vectors to provide for an efficient production of infectious lentivirus vectors. Within the scope of the present invention, arenavirus glycoproteins can be employed in all lentiviral packaging systems for the pseudotyping.

The packaging cells of the invention preferably comprise one or more expression cassettes for the retroviral genes gag, pot, a gene coding for an arenavirus glycoprotein and further a retroviral gene transfer vector for the packaging in the pseudotyped virions which comprises at least one therapeutically applicable transgene and/or marker gene. The packaging cells can further comprise also the genes tat, rev and/or env. Further included are nucleic acid sequences which contain alterations (point mutations, deletions) in the sequences (derivatives). The nucleic acids preferably have a homology of at least 70%, at least 80%, preferably at least 90% or 95% to the original nucleic acids. It is crucial for the use according to the invention that the pseudotyping of retroviral gene transfer particles remains ensured; and also the transduction of the target cells as well as the stable integration of the transgene in the host genome is not impeded. In the following these derivatives should always be included when any gene is mentioned as such.

At the same time the invention also provides pseudotype packaging systems in which one or more further genes of arenaviruses, e.g., LCMV are expressed along with the gp gene product (SEQ ID NO: 4), such as for example the np gene coding for the nucleoprotein (LCMV: coding region for SEQ ID NO: 5; cf. Annex to the sequence protocol, TO SEQ ID NO: 3), the z gene coding for a protein with unknown function (LCMV: coding region for SEQ ID NO: 8; cf. Annex to the sequence protocol, TO SEQ ID NO: 6) and the 1 gene coding for the RNA polymerase (LCMV: coding region for SEQ ID NO: 7; cf. Annex to the sequence protocol, TO SEQ ID NO: 6).

According to a particular embodiment of the invention, these genes can be derived, e.g., from the WE or Armstrong strains of LCMV. In this context, either complete sequences of the genes np, z and/or l (SEQ ID NOs: see above) or parts thereof can be employed.

Therefore, the packaging cells according to the invention can contain in addition to the gp gene of LCMV also at least one gene from the group of LCMV genes consisting of the np gene coding for the nucleoprotein, the l gene coding for the RNA polymerase and the z gene coding for a protein with unknown function.

The packaging cells of the invention and the virions used in the context of the invention comprise at least one therapeutically applicable transgene and/or marker gene. As a therapeutically applicable transgene, a gene is to be understood which can be used in the tumor therapy to directly or indirectly suppress the growth of tumor cells or to kill them. For example, the suicide gene herpes simplex thymidine kinase (HSV-tk) and/or cytosine deaminase can be used. HSV-tk makes the cells transfected with this gene sensitive to gancyclovir. Furthermore, a therapeutically applicable gene can have an immunostimulating effect, such as, e.g., IL-4 or Flt3L. A therapy with B7 or IL-2 can also lead to the regression of tumors as was shown for brain tumors, for example, by T. Lichtor et al. (J. Neurooncol 63 (2003) 247-259). In the therapy of malignant gliomas, success was achieved among others with an expression of antisense DNA against, e.g., TGF-beta (K. Lou, Ann. Med. 36 (2004) 2-8). Further genes which can be used therapeutically for the gene therapy are described, e.g., in Y. Kew et al., Curr. Opin. Neurol. 16 (2003) 665-670. It is understood that also two or more therapeutically applicable genes as well as additional one or more marker genes, if necessary, can be comprised.

Preferred is the marker gene lacZ, an antibiotic resistance gene such as e.g. neo, and/or a gene for a fluorescence protein such as, e.g., eGFP (enhanced green fluorescent protein). The therapeutically applicable transgene and/or marker gene is preferably expressed in the cells of the tumor after the therapy.

The invention further concerns the use of the packaging cells or the virions for the manufacture of a pharmaceutical composition which further comprises suitable accessory agents and/or carriers. The subject-matter of the invention is also a pharmaceutical composition which is suitable for the gene therapy of solid tumors, and comprises the packaging cells which produce vectors pseudotyped with arenavirus glycoprotein and are infiltrating packaging cells and/or are pseudotyped with Lassa virus glycoprotein. The pharmaceutical composition can also comprise suitable accessory agents and/or carriers.

It is preferred that the packaging cells or virions are formulated so that they can be directly introduced into the tumor, for example by injection into the tumor or its direct vicinity. In a brain tumor, this can be done by an intracranial injection. It is, however, also possible to choose an indirect application, as the infiltrating packaging cells migrate specifically into the tumor and the virions possess a high specificity for the tumor cells. For example, the packaging cells or virions can be applied intravenously (i.v.). Especially preferred is an application after tumor resection in which the pharmaceutical composition is given into the resection cavity or in its direct vicinity.

### EXAMPLES

### Example 1

### Generation of Packaging Cells and Virions

Production of packaging cells. Multipotent adult progenitor cells (MAPC) are obtained from rats (Fischer rats, Sprague-Dawley rats) according to known procedures (Y. Jiang et al., Nature 418 (2002) 41-49).

These cells are transfected with the expression vector pGag-Pol-IRES-bsr (S. Morita et al., Gene Therapy 7 (2000) 1063-1066) and selected with blasticidin for ten days. The resulting cell pool stably expresses MoMLVgagpol.

Stable LCMV-GP expression in this gene pool is achieved by transduction with the lentiviral self-inactivating (SIN) vector SEW/GPopt. The lentiviral SIN vector SEW/GPopt was developed on the basis of the vectors pHR′SIN.cPPT-SEW (C. Demaison et al., Hum. Gene Ther. 13 (2002) 803-813) in which the GFP gene was replaced by the codon optimized LCMV-WE-HPIopt. For the production of lentiviral SEW-GPopt vectors, 5 μg SEW/GPopt as well as 5 μg pRSVrev and 15 μg pMDLg/RRE were co-transfected into 293T cells (T. Dull et al., Journal of Virology 72 (1998) 8463-8471). Cell culture supernatants were collected 48 or 72 hours after transfection and were directly used for the transduction of MoMLVgagpol expressing MAPC cell pools. Expression of LCMV-GP in the cell pools was investigated by flow cytometry three days after the transfection.

Flow cytometry analysis of the LCMV-GP expression. For the analysis of the expression of the LCMV glycoprotein, 5×10⁵ cells were harvested, pelleted and incubated with a monoclonal antibody against LCMV-GP1 (M. Bruns et al., Virology 130 (1983) 247-251). After 20 minutes incubation on ice, the cells were washed three times with phosphate-buffered sodium chloride (PBS) and incubated another 20 minutes in a 1:10 dilution of a PE-labeled goat anti-mouse antibody (Dako, Glostrup, Denmark). After three final washings in PBS, the cells were analyzed by a FACSCalibur machine (Becton Dickinson, Heidelberg).

Production of LCMV pseudo-types. To investigate the vector production in stable MoMLVgagpol and LCMV-GPopt expressing cell pools, these were first transduced with a retroviral vector, e.g., MP71EGFP (A. Schambach et al., Molecular Therapy 2 (2000) 435-445).

For this retroviral MP71EGFP vector supernatants were produced by co-transfection of 293T cells with pGag-Pol-IRES-bsr (12 μg), MP71EGFP (7 Ag), pHCMV-LCMV-GP (WE-HPI) (2 μg). The expression plasmid pHCMV-LCMV-GP (WE-HPI) was developed on the basis of the known sequence of the WE-HPI strain (SEQ ID NO: 26; EMBL database accession no. AJ297484) and the pHCMV expression vector (V. Romanowski et al., Virus Res. 3 (1985) 101-114; J. K. Yee, Methods Cell Biol. 43 (1994) 99-112) (W. R. Beyer et al., J. Virol. 75 (2001) 1061-1064; EMBL database accession no. AJ318513).

Five days after transduction of the packaging cells with MP71EGFP, cell culture supernatants of the stably expressing pools were collected and 0.5 ml of each were transferred on 5×10⁴ indicator cells. Expression of the EGFP protein (enhanced green fluorescence protein) in the indicator cells was determined three days later by flow cytometry.

The retroviral vector MP71EGFP was packaged in infectious particles with help of the GagPol and LCMV-GP proteins which are present in the stably expressing gene pools. The MAPC cells are suitable as packaging cells.

### Example 2

### Selective Transduction of Tumors

### Materials and Methods

Cell lines. 9L rat gliosarcoma cell lines, 294 human kidney cell lines and TE671 human fibroblast cell lines were obtained from the American Type Culture Collection. G62 human glioma cells are courtesy of M. Westphal (University Clinic Eppendorf, Germany). The cells were cultured in DMEM supplemented with 10% fetal calf serum and penicillin/streptomycin in a humid atmosphere with 5% CO₂.

Transduction of 9L cells with DsRed. DsRed in a pMP71 vector is courtesy of Norbert Dinauer. For the transduction of the 9L cells with DsRed, 9L cells were seeded in 24-well plates at 5×10⁴ cells/well density. After 4 hours, retroviral supernatants were added for the packaging of the pMP71 DsRed vector. The plates were centrifuged 1 hour at 1000×g. The transduction of the cells was repeated 17 hours after the first transduction. The expression of DsRed was confirmed by fluorescence microscopy (Nicon Eclipse TE300, Dusseldorf, Germany). For the isolation of individual DsRed expressing clones, 100 transduced 9L cells were plated in 10 cm dishes and let produce colonies. DsRed positive colonies were identified with fluorescence microscopy and transferred into separate wells of 24-well plates. The expression level of isolated clones was determined by flow cytometry with a FACS-Calibur machine (Becton Dickinson, San Jose, Calif.). For the in vivo implantation, 1 clone was selected which contained 95% DsRed-positive cells.

Transient production of pseudo-types of lentiviral vectors. Lentiviral vectors were produced by transient transfections of 293T cells. 16 hours before the transfection, 5×10⁶ cells were seeded in 10 cm diameter culture plates in DMEM/FBS. One hour before the transfection, the culture medium was exchanged against 10 ml DMEM/FBS/PS per plate containing 25 μM chloroquin, 50 U penicillin/ml, 50 μg/ml streptomycin (PS, Gibco-Invitrogen). For the transfection of one culture plate, 5 μg pRRL.sinCMVeGFPpre, 5 μg pRSV-Rev, 15 μg pMDLg/pRRE (Dull et al., J. Virol. 72 (1998) 8463-8471) and 1-2 μg of a pHCMV expression plasmid for the glycoprotein of LCMV, pHCMV-LCMV-GP (WE-HPI) (W. R. Beyer et al., J. Virol. 75 (2001) 1061-1064) were used. A mixture containing 450 μl of the plasmid in ddH₂O and 50 μl 2.5 M CaCl₂ was well mixed and then drop by drop added to 500 μl of two-fold HEPES-buffered sodium chloride solution (280 mM NaCl, 100 mM HEPES, 1.5 mM Na₂HPO₄, pH 7.1). After vortexing the sediment was immediately added to the cultures. After 8 hours the medium was exchanged against a 10 ml DMEM/FBS/PS per plate containing 20 mM HEPES. Vector-containing supernatants were collected 24 hours after the transfection and subsequently every 8-16 hours during two days. The cell culture supernatants were pooled and filtered with a MILEX GP filter with 0.22 μm pore size (Millipore, Bedford, Mass.). For in vivo and in vitro (cultured brain cells) applications, the vector supernatants were concentrated by ultra-centrifugation at 19,500 rpm for 2 hours in a SW28 rotor (Beckmann Instruments, California).

Vector titration. Lentiviral vector titers were determined by transduction of different cell lines. Serial dilutions of the cell supernatants were prepared and 0.5 ml of each dilution was added to 5×10⁴ cells and seeded in a well of a 24-well plate 4 hours before the transduction. The plates were centrifuged 1 hour at 1000×g. GFP expressions in the cells was determined 65 hours after the transduction by flow cytometry with a FACSCalibur machine (Becton Dickinson, San Jose, Calif.). The titers were calculated from the dilutions which lead to 0.5% to 20% eGFP positive cells, an interval with linear relation between vector amount and percentage of transduced cells, as multiple integration of vector into the target cells is normally not expected.

Rat hippocampus neuron culture. Primary hippocampus neuron cell cultures were produced as already described (Neumann et al., Science 269 (1995) 549-552). In essence hippocampis from the entire brain of Wistar rats were isolated on day 16 of the embryonic development, the mininges was removed. The cut tissue was dissociated by grinding with a sterile Pasteur pipette. 5×10⁴ cells per ml were given in four-chamber object carriers which were pre-treated with poly-L ornithine (0.5 mg/ml, Sigma, St-Louis, Mo.) in 0.15 M boric acid. The cells were cultured in chemically defined medium which contained basic Eagle medium (BME, Invitrogen, Gaithersburg, Md.) with B27 supplement (2% (v/v), Invitrogen) and glucose (1% (v/v), 45%, Sigma).

Glia cell cultures enriched for rat astrocytes. Hippocampi were isolated from Wistar rats on day 16 of the embryonic development and dissociated into single cell suspensions as described for the neuronal hippocampus preparations. Cells were plated in 50 ml tissue culture flasks which were pre-treated with poly-L lysine (5 μg/ml, Sigma). The cells were cultured in serum which contained medium with MEM-D valine (Invitrogen), 10% heat inactivated FCS (Pan System, Wurzburg, Germany) and 1% L-glutamine. Astrocyte-enriched glia cells were cultured 10-20 days and then plated in BME with B27 supplement (2% (v/v), Invitrogen) and glucose (1% (v/v), 45%, Sigma) into 2×10⁴ cells/ml in four-chamber object carriers before the transduction. It was determined with an immuno-labeling with rabbit antibodies against GFAP (10 μg/ml, Dako, Glostrup, Denmark) that altogether 94% (+3% SC) of the cells were astrocytes.

Transduction of cultured brain cells. 14 days after the plating, neurons and astrocytes were transduced with lentiviral pseudotyped vectors which carried the eGFP marker gene. Two days after the transduction, the cells were fixed in 4% paraformaldehyde and stained with monoclonal mouse anti-beta-tubulin-III antibody (Sigma) for neurons and with monoclonal rabbit anti-GFAP antibody (Dako) for astrocytes. The cells were incubated overnight at 4° C. with the primary antibody. Cy3-goat anti-mouse and Cy3-goat anti-rabbit were used as secondary antibodies for two hours at room temperature. The relative numbers of the transduced (eGFP positive) and non-transduced (eGFP negative) neuronal (beta-tubulin-III positive) and astrocytic (GFAP positive) cells were determined with help of fluorescence microscopy by counting of colonies in 10 chamber fields for each pseudotype.

### Tumor Implantation and Insertion of Lentiviral Vectors

Adult female Fischer 344 rats (Harlan Winkelmann, Borchen, Germany) were anaesthetized by i.p. injection of ketamine (50 mg/kg) and xylazine (2 mg/kg). Intracranial 9L DsRed tumors were established by injection of 1×10⁵ 9L DsRed cells (in 5 μl PBS) into the right striatum using a Hamilton syringe in a stereotactic apparatus (Stoelting, Ill.). The coordinates used were 4 mm lateral opposite to the bregma and in 5 mm depth opposite to the dural surface. 6 days after the tumor implantation, the rats were anaesthetized and lentiviral vector pseudotypes with titers between 2×10⁶ and 1×10⁷ transduced units (TU)/ml were injected using the same stereotactic coordinates and in 1 mm distance (7 different locations). 10 μl total volume was injected into each tumor. Fischer rats which contained no tumor cells were anaesthetized and lentiviral pseudotypes were injected in the right striatum or the right hippocampus. The coordinates used for the hippocampus region were 4.5 mm lateral to the bregma, 5.5 mm behind the coronal plate and in 3 mm depth to the dural surface.

Analysis of rat brains with regard to transduction by lentiviruses. 7 days (tumor-carrying rats) and 14 days (rats without tumor) after the introduction of the lentiviral vector, the animals were euthanized and perfused with 4% paraformaldehyde. The brain was removed, suspended in 30% sucrose for 3 days and then shock-frozen in isopentane filled with liquid nitrogen. Coronal sections (12 μm) were prepared with the cryostat and stained either with rabbit anti-GFAP antibodies (Dako, Hamburg, Germany) for astrocytes or mouse anti-NeuN antibodies (Chemicon, Hofheim, Germany) for neurons. Primary antibodies were incubated overnight at 4° C. Cy3-goat anti-mouse and Cy3-goat anti-rabbit (Dianova, Hamburg, Germany) were used as secondary antibodies for two hours at room temperature. The sections were investigated with a fluorescence microscope (Zeiss, Jena, Germany). The transduction efficiencies in infected tumor regions were estimated (0-10%, 10-50% and 50-100%). In addition, the sections were analyzed with confocal laser scanning microscopy (Leica, UK).

### Results

### Lentiviral Vectors Pseudotyped with LCMV GP and VSV G Transduce 9L Tumor Cells In Vitro

The transduction efficiency of lentiviral vectors pseudotyped with VSV G as well as LCMV GP was compared in vitro in the rat glioma cell lines 9L and 9LDsRed (used tumor implantation). As infection control the human epithelial cell line TE671 was used, which could be transduced with vectors pseudotyped with VSV G as well as LCMV, as shown in a previous study (Beyer et al., 2002, Loc. cit.). End-point dilutions were carried out with 9L, 9LDsRed and TE671 cells; and the percentage of transduced cells was measured using flow cytometry analysis. Both vectors transduced 9L cells, albeit VSV G pseudotypes had a higher efficiency (Table 1). The relative transduction as compared to TE671 was 0.65 for LCMV pseudotypes and 1.91 for VSV G pseudotypes. Otherwise the transduction efficiencies for 9L and 9LDsRed tumor cells in vitro were not significantly different (Table 1).

TABLE 1
Vector titers of pseudo-typed lentiviral vectors
compared to TE671 and glioma cell lines

| | | | VSV G | |
|---|---|---|---|---|
| Cell line | LCMV GP | | | Titer |
| (Number of | Pseudotype titer | Titer compared | Pseudotype titer | compared to |
| experiments) | [TU/ml] (±SD) | to TE671 (±SD) | [TU/ml] (±SD) | TE671 (±SD) |
| TE671 (4) | 2.75 (±1.07) × 10⁴ | 1 | 6.08 (±6.00) × 10⁴ | 1 |
| 9L (4) | 1.80 (±0.78) × 10⁴ | 0.65 (±0.03) | 8.83 (±5.81) × 10⁴ | 1.91 (±0.63) |
| 9LDsRed (2) | 1.29 (±1.00) × 10⁴ | 0.35 (±0.21) | 8.35 (±5.97) × 10⁴ | 1.89 (±0.07) |

Cell lines were transduced with lentiviral vectors pseudotyped with LCMV GP or VSV G which packaged eGFP. The titers were measured by FACS analysis. The results represent average and standard deviations of at least 2 experiments.

### VSV G Pseudotypes Transduced Cultured Neurons and Astrocytes more Efficiently than LCMV GP Pseudotypes.

To analyze the tropism of both pseudotyped vectors for normal brain cells in vitro, cultured neurons and astrocytes which were obtained from 16 days old embryonic Wistar rats by end-point dilutions, were infected. The human glioma cell line G62, which can be infected by both pseudotypes as already shown in a previous study, was used as infection control. VSV G pseudotypes transduced GFAP positive astrocytes and beta-tubulin-III positive neurons at a higher level than LCMV pseudotypes (Table 2). In particular, the transduction efficiency for neurons was clearly different: VSV G pseudotypes transduced 62.5%, whereas LCMV pseudotypes infected only 2.2% of the counted neurons. In addition, LCMV GP pseudotypes show a higher degree of the transduction of the human glioma cell line G62 (89.5%) than that of cultured astrocytes (71.7%) and neurons (2.2%). On the other hand, VSV G pseudotypes transduced astrocytes (85.5%) and neurons (62.5%) at a higher degree than G62 cells (56.9%).

TABLE 2

| | lentiviral pseudotyped | transduced cells/ | total number/ | |
|---|---|---|---|---|
| | vector | high power field | high power field | ratio (%) |
| cultured cells | (titer on TE671) | (±SD) | (±SD) | (±SD) |
| astrocytes | LCMV-GP (8 × 10⁴) | 35.7 (±12.6) | 49.0 (±13.3) | 71.7 (±10.3) |
| | VSV-G (3 × 10⁴) | 43.5 (±18.0) | 49.9 (±16.4) | 85.5 (±12.4) |
| neurons | LCMV-GP (8 × 10⁴) | 0.4 (±0.5) | 17.9 (±5.9) | 2.2 (±3.0) |
| | VSV-G (3 × 10⁴) | 8.2 (±3.3) | 13.8 (±6.1) | 62.5 (±16.9) |
| G62 | LCMV-GP (8 × 10⁴) | 67.3 (±18.3) | 75.5 (±20.3) | 89.5 (±6.7) |
| | VSV-G (3 × 10⁴) | 41.1 (±14.5) | 72.3 (±12.2) | 56.9 (±17.0) |

Cultured rat astrocytes or neurons were transduced with 3×10⁴ to 8×10⁴ eGFP TU of lentiviral vectors pseudotyped with LCMV GP or VSV G. The cells were analyzed by fluorescence microscopy after staining with monoclonal anti-GFAP antibodies against astrocytes or anti-beta-tubulin-III antibodies against neurons. The results represent the average cell numbers and standard deviations from 10 randomly chosen chamber fields.

### VSV G and LCMV GP Pseudotypes Show a Different Tropism Against Normal Brain Cells In Vivo

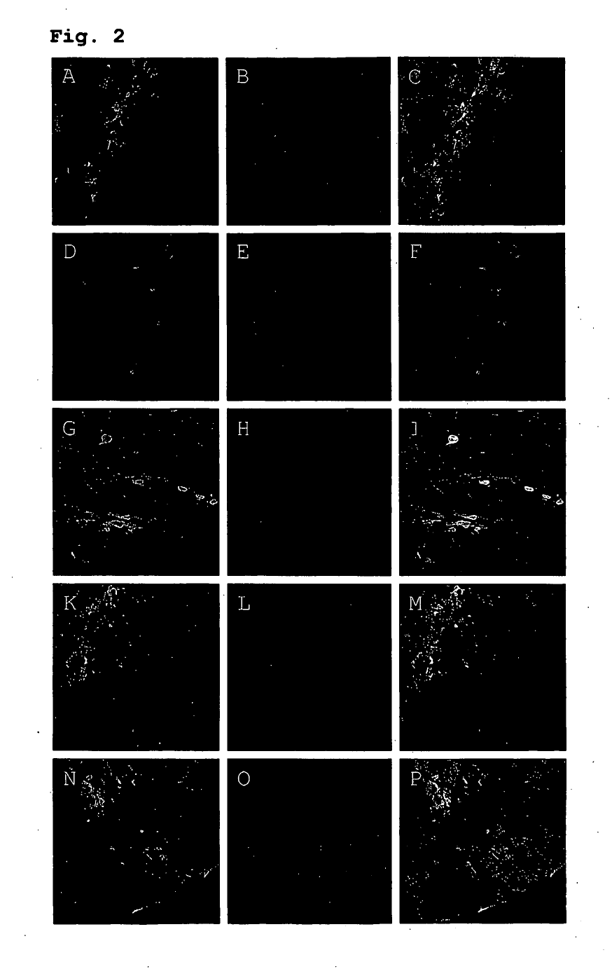

The in vitro results were confirmed in a rat model. For this purpose, LCMV GP and VSV G pseudotypes were injected in striatum and hippocampus of Fischer rats. The relative proportion of the transduced cell types were analyzed by immuno fluorescent staining with cell-specific markers and confocal microscopy. LCMV GP pseudotypes transduced in both brain regions almost exclusively astrocytes, as could be shown by the staining with antibodies against GFAP (FIG. 2A-C). This observation could even be confirmed in regions with higher neuron density (FIG. 2D-F). Transduction of neurons happened rarely. On the other hand, VSV G pseudotypes infected neurons very efficiently; and the estimated ratio of transduced neurons compared to astrocytes was in both brain regions 3:1 (Gig. 2 G-I, K-M, N-P).

### LCMV-GP Pseudotypes Show a Specific and Efficient Transduction of Glial Tumor Cells In Vivo

Finally, the tropism of different pseudotypes was investigated in a rat glioma model. Initially it was tested whether the in vivo growth characteristics of the gene-labeled glioma cell line used for the tumor implantation (9LDsRed) are different from that of the parent cell line 9L. Two weeks after i.c. tumor cell implantation in Fischer rats, the established tumors were investigated histologically with regard to their size and by light (9L) or fluorescence microscopy (9LDsRed) with regard to their ability to penetrate into the brain parenchyma. 9L and 9LDsRed tumors showed no difference in their size, and both infiltrated normal brain to the same extent. With the DsRed marker, even individual tumor cells could be detected which migrated into the brain parenchyma.

To analyze the transduction of the 9LDsRed tumor in vivo by both pseudotypes, two groups of female Fischer rats which carried 9LDsRed tumors were injected with LCMV GP and VSV G pseudotypes. To measure the transduction of the solid tumor and the infiltrating glioma cells, the injections were placed in the center of the tumor and 1 mm away from it. The transduction efficiencies of the infected tumor regions were estimated as described above. LCMV GP pseudotypes showed a very efficient transduction of the solid tumor: 50-100% of the tumor cells were GFP positive in tumor regions injected with the vector supernatants (FIG. 1 A-C). In the infiltrated regions of the 9LDsRed tumors, LCMV GP pseudotypes specifically infected the glioma cells (FIG. 1 D-F). Even individual infiltrating tumor cells were transduced by this vector pseudotype (FIG. 1 E-I). Only a few reactive astrocytes in this region of the tumor bed were GFP positive, as could be shown by staining with antibodies against GFAP. Neurons were not infected in infiltrated tumor regions. VSV G pseudotypes showed a different transduction pattern: solid tumor was transduced to a clearly low extent (0-10%) than with the LCMV GP pseudotypes (FIG. 1 K-M). On the other hand, many normal brain cells including neurons and reactive astrocytes were GFP positive in the infiltrated regions and in the brain parenchyma around it, whereas tumor cells were only rarely transduced (FIG. 1 N-P).

The experiments hence show that intracranial application of lentiviral vectors which were pseudotyped with LCMV glycoprotein leads to specific infection of tumor cells. Although in in vitro experiments and when applying the LCMV GP pseudotyped vectors in brains containing no tumors, astrocytes and—to a lower degree—neurons were infected, an excellent specificity of the transduction with the transgene contained in the pseudotyped vectors can be achieved with the treatment of tumors in vivo.

### Example 3

### Migration Studies

### Materials and Methods

Red fluorescence protein expressing tumor cells (9L RFP) were stereotactically intracranially implanted into Fischer rats (see Example 2 for protocol). On day 5 cells of the cell lines to be investigated were implanted. These cells were transfected with green fluorescence protein (eGFP). On day 8 the brain was removed and a histologic examination was conducted. The following haematopoietic and non-haematopoietic cell lines were investigated in respect of their migration ability:
K562 (human, chronic myeloid leukemia cell line)
U937 (human, monocytes)
Raji (human, B-cell line)
Jurkat (human, T-cell line)
3T3 (murine, fibroblasts)

Also different stem cells which were produced according to the methods known in the art were investigated in respect of their migration ability. Multiple adult progenitor cells (MAPC from Fischer rats, fMAPC) (Young et al., Nature 418 (2002) 41-49), SDMSC (mesenchymal stem cells from Sprague-Dawley rats), as well as murine mesenchymal stem cells (mMSC) (P. Tropelet et al., Exp Cell Res. 2004 May 1; 295(2):395-406) and murine neural stem cells (C17-2 cell line: A. B. Brown et al., Hum Gene Ther. 2003 Dec. 10; 14(18):1777-85) were tested.

The ability of different cells to specifically infiltrate the solid tumor is shown in Table 3.

TABLE 3

| | |
|---|---|
| neural stem cells (NSC), murine | +++ |
| mesenchymal stem cells (MSC), murine and rat | + |
| Multipotent adult progenitor cells (MAPC), rat | +++ |
| (Fischer, Sprague-Dawly) | |
| K562, human chronic myeloid leukemia cell line | − |
| Jurkat, human T-cell line | − |
| Raji, human B-cell line | − |
| U937, human monocyte cell line | − |
| 293, human kidney epithelium cell line | − |
| TE671, human fibroblast cell line | − |
| 3T3, murine fibroblast cell line | − |

+ and − show the ability of the cell type to specifically infiltrate the tumor.

As shown in the Table, the tumor cell lines investigated are not able to infiltrate the tumor. In some cases, some cells remain in the center of the tumor, but become apoptotic. The majority of the cells are located in rings around the tumor; individual cells are, however, detected also in the healthy brain tissue far removed from the tumor. The stem cell lines derived from primary cells are able to infiltrate the tumor. However, among the MSC cells a part of the cells infiltrates the tumor only surface-deep. An optimal infiltration is observed when using MAPC. For NSC, such specific infiltration was already observed before. However, primary NSC cells are only capable of too few cell division ex vivo and must be first genetically transformed (e.g., by SV40 large T) to establish stable lines. NSC cells can hence be employed only after irradiation of the cells. In contrast, MAPC cells can persist for more than 80 cell divisions ex vivo without to transform.

The experiment shows the ability of stem cells to be employed as infiltrating packaging cells in the gene therapy of solid tumors.

### Figure Legends:

### FIG. 1: Solid and Infiltrated Regions of a Rat Glioma were Efficiently Transduced by Lentiviral Vectors Pseudotyped with LCMV GP.

Intracranial 9LDsRed gliomas were infected 7 days after the tumor implantation with lentiviral vectors pseudotyped with LCMV GP or VSV G which express eGFP, and analyzed with confocal laser scanning microscopy on day 14.
(A) 50-100% of the solid tumor was transduced by LCMV GP-pseudotyped vectors.
(B) Solid tumor which expresses DsRed.
(C) Combined pictures of (A) and (B).
(D) Transfection of infiltrated glioma regions by LCMV GP-pseudotyped vectors.
(E) Infiltrated glioma regions which express DsRed.
(F) Combined pictures of (D) and (E).
(G) Individual infiltrating tumor cells transduced by LCMV GP-pseudotyped vectors.
(H) Individual infiltrating tumor cells which express DsRed.
(I) Combined pictures of /G) and (H).
(K) 0-10% of the solid tumor were transduced by VSV G-pseudotyped vectors.
(L) Solid tumor which expresses DsRed.
(M) Combined pictures of (K) and (L).
(N) Infiltrated glioma regions transduced by VSV G-pseudotyped vectors.
(O) Infiltrated glioma regions which express DsRed.
(P) Combined pictures of (N) and (O).
20× magnified.

### FIG. 2: Neurons and Astrocytes were Transduced by Lentiviral Vectors Pseudotyped by VSV G.

Normal rat brain was infected with LCMV GP- or VSV G-pseudotyped vectors which express eGFP. The transduction of neurons and astrocytes was analyzed by confocal laser scanning microscopy after staining with antibodies against NeuN and GFRP on day 14.
(A) Transduction of astrocytes in the striatum by LCMV GP-pseudotyped vectors.
(B) Astrocytes in the striatum which express GFAP.
(C) Combined pictures of (A) and (B).
(D) In regions with high neuron density (hippocampus), no neurons but some astrocytes were transduced.
(E) Hippocampus neurons which express NeuN.
(F) Combined pictures of (D) and (E).
(G) Transduction of hippocampus neurons by VSV G-pseudotyped vectors.
(H) Hippocampus neurons which express NeuN.
(I) Combined pictures of (G) and (H).
(K) Transduction of striatum neurons by VSV. G-pseudotyped vectors.
(L) Striatum neurons which express NeuN.
(M) Combined pictures of (K) and (L).
(N) Hippocampus astrocytes transduced by VSV G-pseudotyped vectors.
(O) Hippocampus astrocytes which express GFAP.
(P) Combined pictures of (N) and (O).
40× magnified.

### Annex to the Sequence Protocol

(Note: The following information is taken from the databank of The National Institute of Health (NIH), U.S.A. The nucleic and amino acid sequences originally contained therein were replaced by the reference to the corresponding SEQ ID NO: (numerical code (400) according to WIPO standard ST 25) of the sequence protocol.)

TO SEQ ID NO: 1

| LOCUS | LCVSRNA 3375 bp ss-RNA VRL 15-JUN-1989 |
|---|---|
| DEFINITION | Lymphocytic choriomeningitis virus S RNA, complete cds. |
| ACCESSION | M22138 |
| NID | g331379 |
| KEYWORDS | S RNA; small RNA. |
| SOURCE | Lymphocytic choriomeningitis virus (strain WE), cDNA to viral |
| | RNA. |
| ORGANISM | Lymphocytic choriomeningitis virus |
| | Viruses; ssRNA negative-strand viruses; Arenaviridae; |
| Arenavirus; | |
| | 1-LCMV-LASV complex. |
| REFERENCE | 1 (bases 1 to 3375) |
| AUTHORS | Romanowski, V., Matsuura, Y. and Bishop, D. H. L. |
| TITLE | Complete sequence of the S RNA of Lymphocytic choriomeningitis |
| | virus (WE strain) compared to that of Pichinde arenavirus |
| JOURNAL | Virus Res. 3, 101-114 (1985) |
| MEDLINE | 86046554 |

| | |
|---|---|
| FEATURES | Location/Qualifiers |
| source | 1..3375 |
| | /organism=“Lymphocytic choriomeningitis virus” |
| | /db_xref=“taxon:11623” |
| CDS | 78..1574 |
| | /note=“S protein” |
| | /codon_start=1 |
| | /db_xref=“PID:g331380” |
| | /translation=SEQ ID NO: 2 |

BASE COUNT 881 a 786 c 725 g 983 t
TO SEQ ID NO: 3

| | |
|---|---|
| LOCUS | LCVGPNP 3376 bp ss-RNA VRL 15-JUN-1989 |
| DEFINITION | Lymphocytic choriomeningitis virus envelope glycoprotein (GP-C) |

and nucleoprotein (NP) genes, complete cds.

| | |
|---|---|
| ACCESSION | M20869 |
| NID | g331358 |
| KEYWORDS | envelope protein; nucleoprotein. |
| SOURCE | Lymphocytic choriomeningitis virus (strain Armstrong 53b), cDNA |
| to viral RNA. | |
| ORGANISM | Lymphocytic choriomeningitis virus |
| | Viruses; ssRNA negative-strand viruses; Arenaviridae; |
| Arenavirus; | |
| | 1-LCMV-LASV complex. |
| REFERENCE | 1 (bases 1 to 3376) |
| AUTHORS | Salvato, M., Shimomaye, E., Southern, P. and Oldstone, M. B. A. |
| TITLE | Virus-lymphocyte interactions: IV. Molecular characterization of |
| | LCMV Armstrong (CTL+) small genomic segment and that of its |
| | variant, clone 13 (CTL−) |
| JOURNAL | Virology 164, 517-522 (1988) |
| MEDLINE | 88219540 |

| | |
|---|---|
| FEATURES | Location/Qualifiers |
| source | 1..3376 |
| | /organism=“Lymphocytic choriomeningitis virus” |
| | /db_xref=“taxon:11623” |
| CDS | 78..1574 |
| | /note=“envelope glycoprotein” |
| | /codon_start=1 |
| | /db_xref=“PID:g331359” |
| | /translation=SEQ ID NO: 4 |
| variation | 856 |
| | /note=“t in ARM 53b; c in ARM 53b Clone 13” |
| variation | 1298 |
| | /note=“t in ARM 53b; c in ARM 53b Clone 13” |
| CDS | complement(1639..3315) |
| | /note=“nucleoprotein” |
| | /codon_start=1 |
| | /db_xref=“PID:g331360” |
| | /translation=SEQ ID NO: 5 |

BASE COUNT 868 a 809 c 748 g 951 t
TO SEQ ID NO: 6

| | |
|---|---|
| LOCUS | LCVLPY 6680 bp ss-RNA VRL 17-MAY-1995 |
| DEFINITION | Lymphocytic choriomeningitis virus putative RNA polymerase L |
| gene, | |
| | complete cds. |
| ACCESSION | J04331 |
| NID | g331368 |
| KEYWORDS | L protein; RNA polymerase. |
| SOURCE | Lymphocytic choriomeningitis virus (strain Armstrong 53b) RNA. |
| ORGANISM | Lymphocytic choriomeningitis virus |
| | Viruses; ssRNA negative-strand viruses; Arenaviridae; |
| | Arenavirus; |
| | 1-LCMV-LASV complex. |
| REFERENCE | 1 (bases 1 to 6680) |
| AUTHORS | Salvato, M., Shimomaye, E. and Oldstone, M. B. |
| TITLE | The primary structure of the lymphocytic choriomeningitis virus |

L gene encodes a putative RNA polymerase

| | |
|---|---|
| JOURNAL | Virology 169 (2), 377-384 (1989) |
| MEDLINE | 89204909 |
| COMMENT | Draft entry and computer-readable sequence of [1] kindly |
| submitted | |
| | by M. Salvato, 18-JAN-1989. |

| | |
|---|---|
| FEATURES | Location/Qualifiers |
| source | 1..6680 |
| | /organism=“Lymphocytic choriomeningitis virus” |
| | /strain=“Armstrong 53b” |
| | /db_xref=“taxon:11623” |
| CDS | 33..6665 |
| | /codon_start=1 |
| | /product=“L protein” |
| | /db_xref=“PID:g331369” |
| | /translation=SEQ ID NO: 7 |
| CDS | complement(6371..6658) |
| | /note=“ORF; putative” |
| | /codon_start=1 |
| | /product=“unknown protein” |
| | /db_xref=“PID:g808709” |
| | /translation=SEQ ID NO: 8 |

BASE COUNT 2065 a 1153 c 1543 g 1919 t
TO SEQ ID NO: 9

| | |
|---|---|
| LOCUS | HUMEF1A 4695 bp DNA PRI 07-NOV-1994 |
| DEFINITION | Human elongation factor EF-1-alpha gene, complete cds. |
| ACCESSION | J04617 J04616 |
| NID | g181962 |
| KEYWORDS | elongation factor. |
| SOURCE | Human placenta DNA, clone pEFG1, and fibroblast cell line GM |
| 637, | |
| | cDNA to mRNA, (library of H. Okayama), clone pAN7. |
| ORGANISM | Homo sapiens |
| | Eukaryotae; mitochondrial eukaryotes; Metazoa; Chordata; |
| | Vertebrata; Eutheria; Primates; Catarrhini; Hominidae; Homo. |
| REFERENCE | 1 (bases 1 to 4695) |
| AUTHORS | Uetsuki, T., Naito, A., Nagata, S. and Kaziro, Y. |
| TITLE | Isolation and characterization of the human chromosomal gene for |
| | polypeptide chain elongation factor-1 alpha |
| JOURNAL | J. Biol. Chem. 264 (10), 5791-5798 (1989) |
| MEDLINE | 89174636 |
| COMMENT | Draft entry and computer-readable sequence for [1] kindly |
| provided | |
| | by S. Nagata, 20-JAN-1989. |

| | |
|---|---|
| FEATURES | Location/Qualifiers |
| source | 1..4695 |
| | /organism=“Homo sapiens” |
| | /db_xref=“taxon:9606” |
| | /map=“Unassigned” |
| misc_binding | 205..214 |
| | /bound_moiety=“Sp1” |
| misc_binding | 320..328 |
| | /bound_moiety=“Sp1” |
| misc_binding | 332..340 |
| | /bound_moiety=“Sp1” |
| TATA_signal | 546..552 |
| prim_transcript | 576..4087 |
| | /note=“EF-1-alpha mRNA and introns” |
| intron | 609..1551 |
| | /note=“EF-1-alpha intron A” |
| misc_binding | 983..992 |
| | /bound_moiety=“Sp1” |
| misc_binding | 1026..1034 |
| | /bound_moiety=“Sp1” |
| misc_binding | 1122..1131 |
| | /bound_moiety=“Sp1” |
| misc_binding | 1132..1141 |
| | /bound_moiety=“Sp1” |
| misc_binding | 1240..1249 |
| | /bound_moiety=“Sp1” |
| misc_binding | 1302..1308 |
| | /bound_moiety=“Ap1” |
| exon | <1582..1725 |
| | /gene=“EEF1A” |
| | /note=“elongation factor EF-1-alpha” |
| | /number=2 |
| gene | join(1582..1725, 2092..2271, 2377..2673, 2757..2907, |
| | 2995..3251, 3341..3575, 3671..3795) |
| | /gene=“EEF1A” |
| CDS | join(1582..1725, 2092..2271, 2377..2673, 2757..2907, |
| | 2995..3251, 3341..3575, 3671..3795) |
| | /gene=“EEF1A” |
| | /note=“elongation factor EF-1-alpha” |
| | /codon_start=1 |
| | /db_xref=“GDB:G00-118-791” |
| | /db_xref=“PID:g181963” |
| | /translation=SEQ ID NO: 10 |
| intron | 1726..2091 |
| | /note=“EF-1-alpha intron B” |
| exon | 2092..2271 |
| | /gene=“EEF1A” |
| | /number=3 |
| intron | 2272..2376 |
| | /note=“EF-1-alpha intron C” |
| exon | 2377..2673 |
| | /gene=“EEF1A” |
| | /number=4 |
| intron | 2674..2756 |
| | /note=“EF-1-alpha intron D” |
| exon | 2757..2907 |
| | /gene=“EEF1A” |
| | /number=5 |
| intron | 2908..2994 |
| | /note=“EF-1-alpha intron E” |
| exon | 2995..3251 |
| | /gene=“EEF1A” |
| | /number=6 |
| intron | 3252..3340 |
| | /note=“EF-1-alpha intron F” |
| exon | 3341..3575 |
| | /gene=“EEF1A” |
| | /number=7 |
| intron | 3576..3670 |
| | /note=“EF-1-alpha intron G” |
| exon | 3671..>3795 |
| | /gene=“EEF1A” |
| | /note=“elongation factor EF-1-alpha” |
| | /number=8 |

BASE COUNT 1200 a 989 c 1235 g 1271 t
TO SEQ ID NO: 11

| | |
|---|---|
| LOCUS | AF033811 8332 bp RNA VRL 05-FEB-1998 |
| DEFINITION | Moloney murine leukemia virus, complete genome. |
| ACCESSION | AF033811 |
| NID | g2801468 |
| KEYWORDS | . |
| SOURCE | Moloney murine leukemia virus. |
| ORGANISM | Moloney murine leukemia virus |
| | Viruses; Retroid viruses; Retroviridae; Mammalian type C |
| | retroviruses; 1-Mammalian type C virus group. |
| REFERENCE | 1 (bases 1 to 8332) |
| AUTHORS | Petropoulos, C. J. |
| TITLE | Appendix 2: Retroviral taxonomy, protein structure, sequences, |
| | and genetic maps |
| JOURNAL | (in) Coffin, J. M. (Ed.); |
| | RETROVIRUSES: 757; |
| | Cold Spring Harbor Laboratory Press, Cold Spring Harbor, New |
| | York, NY, USA (1997) |
| REFERENCE | 2 (bases 1 to 8332) |
| AUTHORS | Chappey, C. |
| TITLE | Direct Submission |
| JOURNAL | Submitted (12-NOV-1997) NIH, NLM, Rockville Pike, Bethesda, MD |
| | 20894, USA |

| | |
|---|---|
| FEATURES | Location/Qualifiers |
| source | 1..8332 |
| | /organism=“Moloney murine leukemia virus” |
| | /db_xref=“taxon:11801” |
| mRNA | 1..8332 |
| | /gene=“gag-pol” |
| mRNA | join(1..205, 5491..8332) |
| | /gene=“env” |
| | /product=“gPr80” |
| 5′UTR | 69..145 |
| gene | 357..1973 |
| | /gene=“gag” |
| CDS | 357..1973 |
| | /gene=“gag” |
| | /codon_start=1 |
| | /product=“Pr65” |
| | /db_xref=“PID:g2801469” |
| | /translation=SEQ ID NO: 12 |
| CDS | join(357..1970, 1974..5573) |
| | /gene=“gag-pol” |
| | /codon_start=1 |
| | /product=“Pr180” |
| | /db_xref=“PID:g2801471” |
| | /translation=SEQ ID NO: 13 |
| mat_peptide | 360..749 |
| | /gene=“gag” |
| | /product=“p15 MA” |
| mat_peptide | 750..1001 |
| | /gene=“gag” |
| | /product=“pp12” |
| mat_peptide | 1002..1790 |
| | /gene=“gag” |
| | /product=“p30 CA” |
| mat_peptide | 1791..1958 |
| | /gene=“gag” |
| | /product=“p10 NC” |
| mat_peptide | join(1959..1970, 1974..2336) |
| | /product=“p14 PR” |
| gene | 1970..5573 |
| | /gene=“pol” |
| mat_peptide | 2337..4349 |
| | /gene=“pol” |
| | /product=“p80 RT” |
| mat_peptide | 4350..5570 |
| | /gene=“pol” |
| | /product=“p46 IN” |
| CDS | 5513..7510 |
| | /gene=“env” |
| | /codon_start=1 |
| | /product=“gPr80” |
| | /db_xref=“PID:g2801470” |
| | /translation=SEQ ID NO: 14 |
| mat_peptide | 5612..6919 |
| | /product=“gp70 SU” |
| mat_peptide | 6920..7507 |
| | /product=“p15E” |
| mat_peptide | 6920..7507 |
| | /product=“p12E TM” |
| 3′UTR | 7818..8264 |

BASE COUNT 2143 a 2395 c 2025 g 1769 t
TO SEQ ID NO: 15

| | |
|---|---|
| LOCUS | HIVNL43 9709 bp ss-RNA VRL 15-JUN-1989 |
| DEFINITION | Human immunodeficiency virus type 1, NY5/BRU (LAV-1) recombinant |
| | clone pNL4-3. |
| ACCESSION | M19921 |
| NID | g328415 |
| KEYWORDS | . |
| SOURCE | Human immunodeficiency virus type 1 (HIV-1), NY5/BRU (LAV-1) |
| | recombinant clone pNL4-3. |
| ORGANISM | Human immunodeficiency virus type 1 |
| | Viruses; Retroid viruses; Retroviridae; Lentivirus; Primate |
| | lentivirus group. |
| REFERENCE | 1 (bases 1 to 9709) |
| AUTHORS | Adachi, A., Gendelman, H. E., Koenig, S., Folks, T., Willey, R., |
| | Rabson, A. and Martin, M. A. |
| TITLE | Production of acquired immunodeficiency syndrome-associated |
| | retrovirus in human and nonhuman cells transfected with an |
| | infectious molecular clone |
| JOURNAL | J. Virol. 59, 284-291 (1986) |
| MEDLINE | 86281827 |
| REFERENCE | 2 (bases 1 to 9709) |
| AUTHORS | Buckler, C. E., Buckler-White, A. J., Willey, R. L. and McCoy, J. |
| JOURNAL | Unpublished (1988) |
| REFERENCE | 3 (sites) |
| AUTHORS | Dai, L. C., Littaua, R., Takahashi, K. and Ennis, F. A. |
| TITLE | Mutation of human immunodeficiency virus type 1 at amino acid |
| | 585 on gp41 results in loss of killing by CD8+ A24-restricted |
| | cytotoxic T lymphocytes |
| JOURNAL | J. Virol. 66, 3151-3154 (1992) |
| MEDLINE | 92219406 |
| COMMENT | [3] sites; revisions of [3]. |
| | Clean copy of sequence [3] kindly provided by Chuck Buckler, |
| | NIAID, Bethesda, MD, 24-JUN-1988. The construction of pNL4-3 |
| | has been described in [1]. pNL4-3 is a recombinant (infectious) |
| | proviral clone that contains DNA from HIV isolates NY5 (5′ half) |
| | and BRU (3′half). The site of recombination is the EcoRI site |
| | at positions 5743-5748. |
| | The length and sequence of the vpr coding region corresponds to |
| | that of the BRU, SC, SF2, MAL and ELI isolates. The vpr coding |
| | region of these isolates is about 18 amino acid residues longer |
| | than the vpr coding region of the IIIb isolates. In HIVNL43, |
| | this shift is due to a single base deletion (with respect to the |
| | IIIb's) at position 5770. The sequence at this position is |
| | ‘atttc’ in HIVNL43 and ‘attttc’ in HIVHXB2. |
| | The original BRU clone, sequenced by Wain-Hobson, et al. (Cell |
| | 40, 9-17 (1985)), and the BRU portion of the pNL4-3 recombinant |
| | clone are different clones from the same BRU isolate. |
| | Two of the revisions reported in the FEATURES produced changes |
| | in amino acid sequences. The revision at position 2421 changes |
| | one amino acid residue from ‘R’ to ‘G’ in the pol coding region. |
| | The revision at positions 8995-9000 changes three amino acid |
| | residues from ‘AHT’ to ‘VTP’ in the nef coding region. |

| | |
|---|---|
| FEATURES | Location/Qualifiers |
| source | 1..9709 |
| | /organism=“Human immunodeficiency virus type 1” |
| | /db_xref=“taxon:11676” |
| LTR | 1..634 |
| | /note=“5′ LTR” |
| repeat_region | 454..550 |
| | /note=“R repeat 5′ copy” |
| prim_transcript | 455..9626 |
| | /note=“tat, rev, nef subgenomic mRNA” |
| intron | 744..5776 |
| | /note=“tat, rev, nef mRNA intron 1” |
| CDS | 790..2292 |
| | /note=“gag polyprotein” |
| | /codon_start=1 |
| | /db_xref=“PID:g328418” |
| | /translation=SEQ ID NO: 16 |
| CDS | 2085..5096 |
| | /partial |
| | /note=“pol polyprotein (NH2-terminus uncertain)” |
| | /codon_start=1 |
| | /db_xref=“PID:g328419” |
| | /translation=SEQ ID NO: 17 |
| CDS | 5041..5619 |
| | /note=“vif protein” |
| | /codon_start=1 |
| | /db_xref=“PID:g328420” |
| | /translation=SEQ ID NO: 18 |
| CDS | 5559..5849 |
| | /note=“vpr protein” |
| | /codon_start=1 |
| | /db_xref=“PID:g328421” |
| | /translation=SEQ ID NO: 19 |
| misc_feature | 5743..5748 |
| | /note=“EcoRI site of recombination” |
| CDS | join(5830..6044, 8369..8414) |
| | /note=“tat protein” |
| | /codon_start=1 |
| | /db_xref=“PID:g328416” |
| | /translation=SEQ ID NO: 20 |
| CDS | join(5969..6044, 8369..8643) |
| | /note=“rev protein” |
| | /codon_start=1 |
| | /db_xref=“PID:g328417” |
| | /translation=SEQ ID NO: 21 |
| intron | 6045..8368 |
| | /note=“tat cds intron 2” |
| intron | 6045..8368 |
| | /note=“tat, rev, nef mRNA intron 2” |
| intron | 6045..8368 |
| | /note=“rev cds intron 2” |
| CDS | 6061..6306 |
| | /note=“vpu protein” |
| | /codon_start=1 |
| | /db_xref=“PID:g328422” |
| | /translation=SEQ ID NO: 22 |
| CDS | 6221..8785 |
| | /note=“envelope polyprotein” |
| | /codon_start=1 |
| | /db_xref=“PID:g328423” |
| | /translation=SEQ ID NO: 23 |
| CDS | 8787..9407 |
| | /note=“nef protein” |
| | /codon_start=1 |
| | /db_xref=“PID:g328424” |
| | /translation=SEQ ID NO: 24 |

1-42. (canceled)

43. A pharmaceutical composition for gene therapy of a solid tumor comprising a packaging cell, wherein said packaging cell comprises a retroviral gene and a gene coding for an arenavirus glycoprotein, and said packaging cell is able to infiltrate a solid tumor.

44. The pharmaceutical composition of claim 43, wherein said packaging cell is a stem cell.

45. The pharmaceutical composition of claim 43, wherein said packaging cell comprises a retroviral gene selected from the group consisting of gag and pol.

46. The pharmaceutical composition of claim 43, wherein said packaging cell comprises a retroviral gene transfer vector for packaging in a pseudotyped retroviral virion.

47. The pharmaceutical composition of claim 46, wherein said retroviral virion comprises a gene selected from the group consisting of a therapeutically applicable gene and a marker gene.

48. The pharmaceutical composition of claim 43, wherein the solid tumor is a brain tumor.

49. The pharmaceutical composition of claim 43, wherein said arenavirus glycoprotein is capable of being produced by a virus selected from the group consisting of lymphocytic choriomeningitis virus (LCMV) and Lassa virus.

50. A pharmaceutical composition for gene therapy of a brain tumor comprising a retroviral virion pseudotyped with an arenavirus glycoprotein.

51. The pharmaceutical composition of claim 50, wherein said arenavirus glycoprotein is capable of being produced by a virus selected from the group consisting of LCMV and Lassa virus.

52. The pharmaceutical composition of claim 50, wherein said retroviral virion comprises a gene selected from the group consisting of a therapeutically applicable gene and a marker gene.

53. The pharmaceutical composition of claim 52, wherein said therapeutically applicable gene is specifically expressed in a cell of said brain tumor following therapy.

54. A packaging cell for gene therapy of a solid tumor comprising a retroviral gene and a gene coding for an arenavirus glycoprotein, wherein said packaging cell is able to infiltrate a solid tumor.

55. The packaging cell of claim 54, wherein said packaging cell comprises a retroviral gene selected from the group consisting of gag and pol.

56. The packaging cell of claim 54, wherein said arenavirus glycoprotein is capable of being produced by a virus selected from the group consisting of LCMV and Lassa virus.

57. A method for treating a solid tumor comprising:
formulating a pharmaceutical composition comprising a packaging cell, wherein said packaging cell comprises a retroviral gene and a gene coding for an arenavirus glycoprotein and said packaging cell is able to infiltrate a solid tumor, and
introducing said pharmaceutical composition in the direct vicinity of the solid tumor.

58. A method for treating a solid tumor comprising:
formulating a pharmaceutical composition comprising a retroviral virion pseudotyped with an arenavirus glycoprotein, and
introducing said pharmaceutical composition in the direct vicinity of the solid tumor.

59. The method of claim 58, wherein the solid tumor is a brain tumor.

60. A method for treating a solid tumor comprising:
formulating a pharmaceutical composition comprising a packaging cell, wherein said packaging cell comprises a retroviral gene and a gene coding for an arenavirus glycoprotein and said packaging cell is able to infiltrate a solid tumor, and
introducing said pharmaceutical composition intravenously.

61. A method for treating a solid tumor comprising:
formulating a pharmaceutical composition comprising a retroviral virion pseudotyped with an arenavirus glycoprotein, and
introducing said pharmaceutical composition intravenously.

62. A method for treating a brain tumor comprising:
formulating a pharmaceutical composition comprising a retroviral virion pseudotyped with an arenavirus glycoprotein, and
injecting said pharmaceutical composition into said brain tumor.


SEQUENCE LISTING (28 sequences)

SEQ ID NO: 1 - DNA, 3375 bp, Lymphocytic choriomeningitis virus
cgcaccggggatcctaggctttttggattgcgctttcctttaggacaactgggtgctggattctatccagtaaaaggatgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttctggctggtaggtcctgtggcatgtacggccttaatggtcccgatatctataaaggggtttaccagttcaaatcagtggagtttgatatgtctcacttaaatctgacgatgcccaatgcgtgctcagtcaacaactctcatcactacatcagtatgggaagctctggactggagccaactttcaccaacgactccatccttaatcacaacttctgcaacttaacctccgctctcaacaaaaagtcttttgaccatacactcatgagtatagtctcgagtctacacctcagtatcagagggaattccaactacaaagcagtgtcttgtgattttaacaatggcatcaccattcaatacaacttgtcatcttcggacccacagagcgccatgagccagtgtaggactttcagaggtagagtcttggacatgtttagaactgcctttggaggaaagtacatgagaagtggctggggctggacaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagactttcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggctgccctgagtaagttcaagcaagatgtagagtctgccttgcatgtattcaaaacaacattaaattctctgatttccgatcagctgttgatgaggaatcatctaagagatctaatgggggtaccatactgtaattactcaaagttctggtatctggaacatgctaagactggtgagactagtgtacccaagtgttggcttgtcactaatggctcctacttgaatgagacccattttagtgatcaaatcgaacaagaagcagataacatgatcacagagatgttgaggaaggactacataaaaagacaagggagtactcctttagccttaatggatcttttgatgttttcaacatcagcatacttgatcagcatctttctgcattttgtgaggataccaacacatagacacataaagggcggttcatgtccaaagccacatcgcttgaccaacaaggggatctgtagttgtggtgcattcaaggtgcctggtgtaaaaactatctggaaaagacgctgatcagcagcgcctccctgactctccacctcgaaagaggtggagagtcagggaggcccagcgggtcttagagtgtcacaacattgggtcctctgaagatcaaatcatgtggcaggatgttgtgaacggtctttagatcagggagtcttgccttggaagcactctcaaagatgatgcagtccatgagtgcacagtgtggggtgatttctttcttctttttgtctctcactaccccagtgtgcattttgcatagccagccatatttgtcccacactttatcttcatattctcttgaggcctccttagtcatctcaacatcaatgagttttatgtcccttctattctgtgagtccagaagctttctgatgtcatcagaaccttgacagctcaagaccatcccttgtgggagagcacctataactgatgaggtcagcccagcctgtgcattgaagaggtcagcaagatccatgccgtgtgaatacttggagtcctgcttgaattgcttctggtccgtaggttctctgtaaaaatgtatgaattgcccattttgtggttgaaatattgctatctccactggatcattgaacctgccttcaatgtcaatccatgtgggagcattgggatcaatccctcccatcaagtctttcaacagcattgtttgactgtaactcaagcccacctgaggtgggcctgctgctccaggcactggcctagatgagttggccacaagtttttcatttgtgagatcaattgtcgtgttctcccatgctctccccacaactgacgttctacaggctatgtatggccatccttcacctgaaagacagactttataaaggatgttttcataaggatttctatccccaacttgatctgagacaaacatgttgagtttcttcttggccccaaggactgcttttaggagatcctcactattgcttggtttgatcaaaatagattccagcatgttccctccatgtagcagagctgcccccgctttcacagccgcaccaagactgaaattataaccagagatattgatactagattgctgttcagtaatgacccccagaactgggtgtttatcttttagcctttctaggtcactgagattcgggtatttgactgtgtaaagtaagccaaggtctgtgagtgcctgcacaacatcattgagtggggtctgtgactgttttgccatgcaagccattgtcaggcttggcattgtgccgaactgattgttcagaagtgatgagtccttcacatcccaaacccttactacaccacttgcaccctgctgaggtcttctcatcccaaccatttgcagtatttgggatctctgatcaagttgttgtgctgtcaaatttcccatgtagactccagaagcttgaggcctctcagttctcataattttggccttcagcttctcaagatcagctgcaagggtcatcaattcctctgcactaagtcttcccactttcagaacatttttctttgatgtagacttcggatcaacaagagaatgcacagtctggttaagactcctgagtctctgcaagtctttatcgtccctcctttcctttctcatgatcctctgaacgttgctgacttcagaaaagtccaacccatttagaagactggttgcgtccttgatgacggcagcctttacatctgatgtaaaaccctgcaactccctcctcaacgcctgtgtccactgaaagcttttgacttctttggacaaagacattttgtcacacaatgaatttccaaataaaagcgcaatcaaatgcctaggatccactgtgcg

SEQ ID NO: 2 - protein, 498 aa, Lymphocytic choriomeningitis virus
MGQIVTMFEALPHIIDEVINIVIIVLIIITSIKAVYNFATCGILALVSFLFLAGRSCGMYGLNGPDIYKGVYQFKSVEFDMSHLNLTMPNACSVNNSHHYISMGSSGLEPTFTNDSILNHNFCNLTSALNKKSFDHTLMSIVSSLHLSIRGNSNYKAVSCDFNNGITIQYNLSSSDPQSAMSQCRTFRGRVLDMFRTAFGGKYMRSGWGWTGSDGKTTWCSQTSYQYLIIQNRTWENHCRYAGPFGMSRILFAQEKTKFLTRRLSGTFTWTLSDSSGVENPGGYCLTKWMILAAELKCFGNTAVAKCNVNHDEEFCDMLRLIDYNKAALSKFKQDVESALHVFKTTLNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHFVRIPTHRHIKGGSCPKPHRLTNKGICSCGAFKVPGVKTIWKRR

SEQ ID NO: 3 - DNA, 3376 bp, Lymphocytic choriomeningitis virus
cgcaccggggatcctaggctttttggattgcgctttcctctagatcaactgggtgtcaggccctatcctacagaaggatgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctgaagaacagcgcctccctgactctccacctcgaaagaggtggagagtcagggaggcccagagggtcttagagtgtcacaacatttgggcctctaaaaattaggtcatgtggcagaatgttgtgaacagttttcagatctgggagccttgctttggaggcgctttcaaaaatgatgcagtccatgagtgcacagtgcggggtgatctctttcttctttttgtcccttactattccagtatgcatcttacacaaccagccatatttgtcccacactttgtcttcatactccctcgaagcttccctggtcatttcaacatcgataagcttaatgtccttcctattctgtgagtccagaagctttctgatgtcatcggagccttgacagcttagaaccatcccctgcggaagagcacctataactgacgaggtcaacccgggttgcgcattgaagaggtcggcaagatccatgccgtgtgagtacttggaatcttgcttgaattgtttttgatcaacgggttccctgtaaaagtgtatgaactgcccgttctgtggttggaaaattgctatttccactggatcattaaatctaccctcaatgtcaatccatgtaggagcgttggggtcaattcctcccatgaggtcttttaaaagcattgtctggctgtagcttaagcccacctgaggtggacctgctgctccaggcgctggcctgggtgaattgactgcaggtttctcgcttgtgagatcaattgttgtgttttcccatgctctccccacaatcgatgttctacaagctatgtatggccatccttcacctgaaaggcaaactttatagaggatgttttcataagggttcctgtccccaacttggtctgaaacaaacatgttgagttttctcttggccccgagaactgccttcaagaggtcctcgctgttgcttggcttgatcaaaattgactctaacatgttacccccatccaacagggctgcccctgccttcacggcagcaccaagactaaagttatagccagaaatgttgatgctggactgctgttcagtgatgacccccagaactgggtgcttgtctttcagcctttcaagatcattaagatttggatacttgactgtgtaaagcaagccaaggtctgtgagcgcttgtacaacgtcattgagcggagtctgtgactgtttggccatacaagccatagttagacttggcattgtgccaaattgattgttcaaaagtgatgagtctttcacatcccaaactcttaccacaccacttgcaccctgctgaggctttctcatcccaactatctgtaggatctgagatctttggtctagttgctgtgttgttaagttccccatatatacccctgaagcctggggcctttcagacctcatgatcttggccttcagcttctcaaggtcagccgcaagagacatcagttcttctgcactgagcctccccactttcaaaacattcttctttgatgttgactttaaatccacaagagaatgtacagtctggttgagacttctgagtctctgtaggtctttgtcatctctcttttccttcctcatgatcctctgaacattgctgacctcagagaagtccaacccattcagaaggttggttgcatccttaatgacagcagccttcacatctgatgtgaagctctgcaattctcttctcaatgcttgcgtccattggaagctcttaacttccttagacaaggacatcttgttgctcaatggtttctcaagacaaatgcgcaatcaaatgcctaggatccactgtgcg

SEQ ID NO: 4 - protein, 498 aa, Lymphocytic choriomeningitis virus
  MISC_FEATURE (1)..(498): envelope glycoprotein
MGQIVTMFEALPHIIDEVINIVIIVLIVITGIKAVYNFATCGIFALISFLLLAGRSCGMYGLKGPDIYKGVYQFKSVEFDMSHLNLTMPNACSANNSHHYISMGTSGLELTFTNDSIISHNFCNLTSAFNKKTFDHTLMSIVSSLHLSIRGNSNYKAVSCDFNNGITIQYNLTFSDRQSAQSQCRTFRGRVLDMFRTAFGGKYMRSGWGWTGSDGKTTWCSQTSYQYLIIQNRTWENHCTYAGPFGMSRILLSQEKTKFFTRRLAGTFTWTLSDSSGVENPGGYCLTKWMILAAELKCFGNTAVAKCNVNHDAEFCDMLRLIDYNKAALSKFKEDVESALHLFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLVSIFLHLVKIPTHRHIKGGSCPKPHRLTNKGICSCGAFKVPGVKTVWKRR

SEQ ID NO: 5 - protein, 558 aa, Lymphocytic choriomeningitis virus
  MISC_FEATURE (1)..(558): nucleoprotein
MSLSKEVKSFQWTQALRRELQSFTSDVKAAVIKDATNLLNGLDFSEVSNVQRIMRKEKRDDKDLQRLRSLNQTVHSLVDLKSTSKKNVLKVGRLSAEELMSLAADLEKLKAKIMRSERPQASGVYMGNLTTQQLDQRSQILQIVGMRKPQQGASGVVRVWDVKDSSLLNNQFGTMPSLTMACMAKQSQTPLNDVVQALTDLGLLYTVKYPNLNDLERLKDKHPVLGVITEQQSSINISGYNFSLGAAVKAGAALLDGGNMLESILIKPSNSEDLLKAVLGAKRKLNMFVSDQVGDRNPYENILYKVCLSGEGWPYIACRTSIVGRAWENTTIDLTSEKPAVNSPRPAPGAAGPPQVGLSYSQTMLLKDLMGGIDPNAPTWIDIEGRFNDPVEIAIFQPQNGQFIHFYREPVDQKQFKQDSKYSHGMDLADLFNAQPGLTSSVIGALPQGMVLSCQGSDDIRKLLDSQNRKDIKLIDVEMTREASREYEDKVWDKYGWLCKMHTGIVRDKKKKEITPHCALMDCIIFESASKARLPDLKTVHNILPHDLIFRGPNVVTL

SEQ ID NO: 6 - DNA, 6680 bp, Lymphocytic choriomeningitis virus
cgcaccgaggatcctaggctttttgatgcgcaatggatgaaatcatctcagaattgagagagttatgtttaaactatatagaacaggatgagaggttgtcaaggcagaaactcaactttctgggacaaagggaacccagaatggttctgattgagggactcaagttgctgtcacgctgcattgaaatagacagtgcagacaagagtggctgcacacacaaccacgacgataagtctgtggaaacaattttggtggagtctggaattgtatgcccaggactaccacttatcattcctgatggttacaagctgatagacaattctctcattcttcttgagtgttttgttaggagctcaccagccagttttgagaagaaatttatagaggacactaacaaattggcatgcatcagggaagaccttgctgttgcgggtgtcacattagttccaatagtagatggtcgttgtgattatgataatagttttatgccagagtgggcaaacttcaaatttagagaccttttattcaaacttttggagtattctaaccaaaatgagaaagtctttgaagagtctgaatattttagactctgtgagtccctgaagactactatcgacaagcgctccggtatggactctatgaaaattctgaaagatgcgaggtcaactcacaatgatgaaattatgaggatgtgccacgaaggcatcaaccccaacatgagctgtgatgatgtggtttttggaataaactctcttttcagcaggtttagaagagatttagaaagtgggaaattaaagagaaactttcagaaagtaaaccctgaaggcttgatcaaggaattctctgagctctatgaaaaccttgctgatagtgatgatatcttaacattaagcagggaggcagtcgaatcctgtcctttgatgagattcataactgcagagacccatgggcacgaaaggggaagtgagactagcactgaatatgagaggctcctctctatgttaaacaaagtcaagagtttgaaactgttgaatactagaaggagacagttgttaaatctggatgttttgtgtctttcctcattgataaaacagtcgaaattcaaagggttaaaaaatgataaacactgggtgggttgttgctatagtagtgtgaatgataggctggtaagctttcacagcactaaagaggagttcattagacttttgaggaatagaaaaaagtcaaaggtgtttagaaaggtgtcttttgaggaattgtttagggcgtctattagtgagttcattgcaaaaattcaaaaatgcctgttagtggtgggactgagtttcgagcattacggactgtctgaacaccttgagcaagaatgccacataccattcactgaatttgagaactttatgaaaattggagctcacccgataatgtattatacgaagtttgaagattacaatttccaacccagcacagagcagctgaagaacatacagagcctgagaagattatcatctgtttgtctggccttaacaaacagtatgaaaactagctcagttgctagactaaggcaaaatcaaatagggtctgtgagatatcaagtggtagaatgcaaagaagtgttttgccaagtaataaaactggactctgaagaataccacctattataccagaagactggagaatcttcaaggtgctactccatacaaggcccggatggtcatttaatttccttctatgcagatcctaaaaggttctttttaccaattttttcagatgaggtcttatacaatatgatagacatcatgatttcatggattagatcatgtcctgatttgaaagactgtctcaccgacattgaggttgcactgaggaccctattgttgctaatgctcaccaacccaacaaagagaaatcaaaagcaggtacagagtgtcagatatttggtgatggcaatagtgtcagatttttcatctacatcattaatggataagttgagggaggatctgatcacacctgctgagaaggtggtgtataagctgcttagattcctaataaaaactatttttggtactggtgagaaggtgttgttgagtgcaaaatttaaatttatgttgaatgtgtcatacctgtgtcatttgatcacaaaggagacccctgacaggctaacagatcagataaaatgttttgaaaagttctttgagcccaaaagtcaatttggtttttttgtcaaccccaaggaagcaatcactcctgaggaagaatgtgtgttctatgagcaaatgaagagattcactagtaaagaaattgactgtcagcatacaactccaggtgttaatctggaagcctttagcctaatggtgtcttcatttaacaacggcactttaattttcaaaggagagaagaagctaaacagcctagatcccatgactaactctggatgtgcgacagcattagatcttgctagtaacaaaagtgtggtggttaataagcatctaaatggagaacgacttctggaatatgactttaacaaattgcttgttagtgctgtgagtcaaattacggagagtttcgtaagaaaacaaaagtataagttgagccactcagactatgaatataaagtttccaagttagtctctagattggtcatcggttccaagggagaagagacagggagatcggaagacaacctggcagaaatatgttttgatggagaagaagagacaagcttcttcaaaagtctcgaagaaaaggtcaacaccacaatagcacggtacagaagaggtaggagggccaatgacaaaggagatggagaaaaacttacaaatacaaaaggactacatcatttacagcttattctaacagggaagatggctcacttaagaaaagttatcttgtcagaaatatctttccatttagtagaagactttgacccatcatgtctaaccaatgatgacatgaaatttatctgtgaggctgttgagggttccacagagctgtcacctttgtatttcacctcagtcattaaagatcagtgtggcctcgatgagatggcaaaaaacctttgtagaaagttcttttctgagaatgattggttttcttgcatgaagatgattctgttgcaaatgaatgcaaatgcgtactcagggaaatacaggcatatgcaaaggcaaggcttgaatttcaaatttgactgggacaaactggaagaagacgtgagaatcagtgagagggaaagtaattctgagtcccttagtaaagctctgtcgttgacaaaatgtatgagtgctgctttgaaaaatctgtgcttctactcagaagaatcaccaacatcatacacctcagtaggtcctgactctggaaggctgaaatttgcactatcttataaagagcaggttgggggaaatagagaactctatattggagatttgaggacaaaaatgttcacaaggttaatagaagattattttgagtctttttcaagtttcttttcaggctcctgtttaaacaatgataaggaatttgaaaatgcaatcttgtcaatgactatcaatgtgcgggaagggttcttaaactatagtatggatcacagcaaatggggaccaatgatgtgcccatttttgttcttaatgtttctacaaaatctcaaactaggtgatgaccagtatgtgcgttccgggaaagatcatgttagcactttgttaacttggcacatgcataagcttgtcgaggtcccctttcctgttgtgaatgcaatgatgaaatcatatgtcaagtcgaagctaaaacttctcaggggttcagaaacaactgttactgagagaattttcagacaatattttgaaatggggatagtgccatcccatatatccagccttattgatatggggcagggaatcttgcataatgcttctgacttctatggtttgcttagcgagaggttcatcaactactgcattggtgttatctttggcgaaagaccagaggcttacacatcaagtgatgatcagatcactttatttgataggaggctgagtgacctggttgtaagtgatccggaggaagtccttgtcctgttggaattccaatctcatctgagcggcttgttaaacaaatttatcagcccaaaaagtgtggctgggaggttcgctgcagaatttaaatctagattctatgtatggggggaggaagtccctcttctcacaaagtttgtatctgcagcgctacacaatgtcaagtgtaaagagccacatcaactttgtgaaacaatagatacaattgcagatcaagccatcgcaaatggcgtcccagtctccctagttaatagtatccaaaggagaacactggacctcctaaagtatgccaatttccctttggatccatttctactgaataccaacactgatgtgaaagattggctggatggttctagaggttacagaatacaaagactcattgaggaactgtgtcctaatgaaacaaaggttgtaagaaagcttgtaaggaaactgcatcataagctcaaaaatggtgaatttaatgaagaatttttcttagacctatttaacagagataaaacggaggccattcttcaattgggagacctcctcggtcttgaagaagatctgaatcagttagcagatgttaactggttgaatttgaatgaaatgttcccattaaggatggttttaagacaaaaggtggtttatccatcagtgatgactttccaagaggaaagaatcccatcattgatcaagacactccagaacaaactttgtagtaaattcacaaggggtgcacagaagctgctgtcagaagcaatcaacaagtcagctttccagagttgtatctcatctggctttataggcctttgcaaaactctaggaagcaggtgtgtgagaaacaaaaatagggaaaatctgtatatcaaaaagctgcttgaggatctaaccacagatgatcatgtgacaagagtttgcaatcgggatggtataacgctgtacatttgtgacaaacagtctcatccagaagcccaccgtgatcatatatgccttttaaggcctcttctttgggactacatttgtatttcattgagcaactcttttgagttgggtgtttgggtcctagcagaaccgaccaaagggaagaataacagtgagaacctaactcttaagcacttaaacccatgtgattatgtagcaagaaagcctgagagctcaaggctactggaggacaaagtgaatttgaaccaagtgattcaatctgtgaggcggctatatcccaagatctttgaggatcagcttcttccatttatgtctgacatgagctcaaaaaacatgaggtggagtcccagaattaaattccttgacctctgtgttttaattgatattaactcagaatccttgtcactcatttctcatgttgttaagtggaaaagggatgaacattacactgttctgttttctgaccttgccaattctcatcagcgatctgactccagtctggttgatgaatttgttgttagcacgagggatgtctgcaagaacttcttaaaacaggtgtattttgaatcatttgttcgagaatttgttgcaacaaccaggacattaggcaatttttcatggttccctcataaagaaatgatgccatctgaagatggtgctgaggcactgggcccctttcaatcatttgtctcaaaggtggtgaacaaaaatgtggagaggcctatgtttaggaatgatttgcagtttggttttgggtggttctcttaccgaatgggagatgttgtgtgtaatgctgccatgttgattaggcagggcctgacaaacccaaaggcatttaaatccttaaaggatctgtgggactacatgctcaactacacaaaaggggtattggagttttcaatttcagtggactttacgcacaatcagaataatactgactgtttaaggaaattttcattgatattcttggttaggtgccaattacagaatccaggtgtggctgaacttttatcatgctctcacctctttaagggtgagatagatagaagaatgttggatgaatgcctccacttactgaggacagactctgtcttcaaggtgaacgatggtgtctttgatatcagatctgaagagtttgaggattacatggaagatcccttgatacttggtgattctcttgagcttgagttgttgggctccaaaagaatactggatgggattagatctattgactttgagagagttggacctgagtgggagcctgtgccactgactgtaaagatgggtgccctttttgaaggaagaaaccttgtccaaaatatcattgtgaagctggagaccaaggacatgaaagtctttctagcaggacttgagggctatgaaaagattagtgatgtccttgggaacctcttcctgcatcgattcagaactggtgaacatttgttgggttcagagataagtgtaatcctccaggaactatgtatagacagatctattctgctgattccactgtcgcttttgccagactggttcgcctttaaggattgcagactttgttttagcaaatctaggagcactttgatgtatgaaatagtggggggcaggtttagactcaaggggaggtcctgcgacgattggctaggcgggtcggtggccgaggacatcgactgatgggcatctcctggg

SEQ ID NO: 7 - protein, 2210 aa, Lymphocytic choriomeningitis virus
  MISC_FEATURE (1)..(2210): L-protein
MDEIISELRELCLNYIEQDERLSRQKLNFLGQREPRMVLIEGLKLLSRCIEIDSADKSGCTHNHDDKSVETILVESGIVCPGLPLIIPDGYKLIDNSLILLECFVRSSPASFEKKFIEDTNKLACIREDLAVAGVTLVPIVDGRCDYDNSFMPEWANFKFRDLLFKLLEYSNQNEKVFEESEYFRLCESLKTTIDKRSGMDSMKILKDARSTHNDEIMRMCHEGINPNMSCDDVVFGINSLFSRFRRDLESGKLKRNFQKVNPEGLIKEFSELYENLADSDDILTLSREAVESCPLMRFITAETHGHERGSETSTEYERLLSMLNKVKSLKLLNTRRRQLLNLDVLCLSSLIKQSKFKGLKNDKHWVGCCYSSVNDRLVSFHSTKEEFIRLLRNRKKSKVFRKVSFEELFRASISEFIAKIQKCLLVVGLSFEHYGLSEHLEQECHIPFTEFENFMKIGAHPIMYYTKFEDYNFQPSTEQLKNIQSLRRLSSVCLALTNSMKTSSVARLRQNQIGSVRYQVVECKEVFCQVIKLDSEEYHLLYQKTGESSRCYSIQGPDGHLISFYADPKRFFLPIFSDEVLYNMIDIMISWIRSCPDLKDCLTDIEVALRTLLLLMLTNPTKRNQKQVQSVRYLVMAIVSDFSSTSLMDKLREDLITPAEKVVYKLLRFLIKTIFGTGEKVLLSAKFKFMLNVSYLCHLITKETPDRLTDQIKCFEKFFEPKSQFGFFVNPKEAITPEEECVFYEQMKRFTSKEIDCQHTTPGVNLEAFSLMVSSFNNGTLIFKGEKKLNSLDPMTNSGCATALDLASNKSVVVNKHLNGERLLEYDFNKLLVSAVSQITESFVRKQKYKLSHSDYEYKVSKLVSRLVIGSKGEETGRSEDNLAEICFDGEEETSFFKSLEEKVNTTIARYRRGRRANDKGDGEKLTNTKGLHHLQLILTGKMAHLRKVILSEISFHLVEDFDPSCLTNDDMKFICEAVEGSTELSPLYFTSVIKDQCGLDEMAKNLCRKFFSENDWFSCMKMILLQMNANAYSGKYRHMQRQGLNFKFDWDKLEEDVRISERESNSESLSKALSLTKCMSAALKNLCFYSEESPTSYTSVGPDSGRLKFALSYKEQVGGNRELYIGDLRTKMFTRLIEDYFESFSSFFSGSCLNNDKEFENAILSMTINVREGFLNYSMDHSKWGPMMCPFLFLMFLQNLKLGDDQYVRSGKDHVSTLLTWHMHKLVEVPFPVVNAMMKSYVKSKLKLLRGSETTVTERIFRQYFEMGIVPSHISSLIDMGQGILHNASDFYGLLSERFINYCIGVIFGERPEAYTSSDDQITLFDRRLSDLVVSDPEEVLVLLEFQSHLSGLLNKFISPKSVAGRFAAEFKSRFYVWGEEVPLLTKFVSAALHNVKCKEPHQLCETIDTIADQAIANGVPVSLVNSIQRRTLDLLKYANFPLDPFLLNTNTDVKDWLDGSRGYRIQRLIEELCPNETKVVRKLVRKLHHKLKNGEFNEEFFLDLFNRDKTEAILQLGDLLGLEEDLNQLADVNWLNLNEMFPLRMVLRQKVVYPSVMTFQEERIPSLIKTLQNKLCSKFTRGAQKLLSEAINKSAFQSCISSGFIGLCKTLGSRCVRNKNRENLYIKKLLEDLTTDDHVTRVCNRDGITLYICDKQSHPEAHRDHICLLRPLLWDYICISLSNSFELGVWVLAEPTKGKNNSENLTLKHLNPCDYVARKPESSRLLEDKVNLNQVIQSVRRLYPKIFEDQLLPFMSDMSSKNMRWSPRIKFLDLCVLIDINSESLSLISHVVKWKRDEHYTVLFSDLANSHQRSDSSLVDEFVVSTRDVCKNFLKQVYFESFVREFVATTRTLGNFSWFPHKEMMPSEDGAEALGPFQSFVSKVVNKNVERPMFRNDLQFGFGWFSYRMGDVVCNAAMLIRQGLTNPKAFKSLKDLWDYMLNYTKGVLEFSISVDFTHNQNNTDCLRKFSLIFLVRCQLQNPGVAELLSCSHLFKGEIDRRMLDECLHLLRTDSVFKVNDGVFDIRSEEFEDYMEDPLILGDSLELELLGSKRILDGIRSIDFERVGPEWEPVPLTVKMGALFEGRNLVQNIIVKLETKDMKVFLAGLEGYEKISDVLGNLFLHRFRTGEHLLGSEISVILQELCIDRSILLIPLSLLPDWFAFKDCRLCFSKSRSTLMYEIVGGRFRLKGRSCDDWLGGSVAEDID

SEQ ID NO: 8 - protein, 95 aa, Lymphocytic choriomeningitis virus
  MISC_FEATURE (1)..(95): unknown protein
MSSATDPPSQSSQDLPLSLNLPPTISYIKVLLDLLKQSLQSLKANQSGKSDSGISRIDLSIHSSWRITLISEPNKCSPVLNRCRKRFPRTSLIFS

SEQ ID NO: 9 - DNA, 4695 bp, Homo sapiens
cccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgaggtatgtttaataccagaaagggaaagatcaactaaaatgagttttaccagcagaatcattaggtgatttccccagaactagtgagtggtttagatctgaatgctaatagttaagaccttacttatgaaataattttgcttttggtgacttctgtaatcgtattgctagtgagtagatttggatgttaatagttaagatcctacttataaaagtttgatttttggttgcttctgtaacccaaagtgaccaaaatcactttggacttggagttgtaaagtggaaactgccaattaagggctggggacaaggaaattgaagctggagtttgtgttttagtaaccaagtaacgactcttaatccttacagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggttgggattaataattctaggtttctttatcccaaaaggcttgctttgtacactggttttgtcatttggagagttgacagggatatgtctttgctttctttaaaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacgtaagtggctttcaagaccattgttaaaaagctctgggaatggcgatttcatgcttacacaaattggcatgcttgtgtttcagatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtaagttggctgtaaacaaagttgaatttgagttgatagagtactgtctgccttcataggtatttagtatgctgtaaatatttttaggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtaacaatttaaagtaacattaacttattgcagaggctaaagtcatttgagactttggatttgcactgaatgcaaatcttttttccaaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtaaggatgactacttaaatgtaaaaaagttgtgttaaagatgaaaaatacaactgaacagtactttgggtaataattaactttttttttaataggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggtttctttgattacaacactggagagaaatgcagcatgttgctgattgcctgtcactaaaacaggccaaaaactgagtccttgggttgcatagaaagcttcatgttgctaaaccaatgttaagtgaatctttggaaacaaaatgtttccaaattactgggatgtgcatgttgaaacgtgggttaaaatgactgggcagtgaaagttgactatttgccatgacataagaaataagtgtagtggctagtgtacaccctatgagtggaagggtccattttgaagtcagtggagtaagctttatgccattttgatggtttcacaagttctattgagtgctattcagaataggaacaaggttctaatagaaaaagatggcaatttgaagtagctataaaattagactaattacattgcttttctccgac

SEQ ID NO: 10 - protein, 462 aa, Homo sapiens
  MISC_FEATURE (1)..(462): elongation factor EF-1-alpha
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 11 - DNA, 8332 bp, Moloney murine leukemia virus
tatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgagaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctacacctcagaacattttcattacacagtgactgatataaaggacctaaccaagttgggggccatttatgataaaacaaagaagtattgggtctaccaaggaaaacctgtgatgcctgaccagtttacttttgaattattagactttcttcatcagctgactcacctcagcttctcaaaaatgaaggctctcctagagagaagccacagtccctactacatgctgaaccgggatcgaacactcaaaaatatcactgagacctgcaaagcttgtgcacaagtcaacgccagcaagtctgccgttaaacagggaactagggtccgcgggcatcggcccggcactcattgggagatcgatttcaccgagataaagcccggattgtatggctataaatatcttctagtttttatagataccttttctggctggatagaagccttcccaaccaagaaagaaaccgccaaggtcgtaaccaagaagctactagaggagatcttccccaggttcggcatgcctcaggtattgggaactgacaatgggcctgccttcgtctccaaggtgagtcagacagtggccgatctgttggggattgattggaaattacattgtgcatacagaccccaaagctcaggccaggtagaaagaatgaatagaaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgacatggcgcgttcaacgctctcaaaaccccttaaaaataaggttaacccgcgaggccccctaatccccttaattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtgggactcctctctcccctacccaacttccaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcagcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattt

SEQ ID NO: 12 - protein, 538 aa, Moloney murine leukemia virus
  MISC_FEATURE (1)..(538): gag protein
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 13 - protein, 1737 aa, Moloney murine leukimia virus
  MISC_FEATURE (1)..(1737): gag-pol protein
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVMGPMGQPLQVLTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPYTSEHFHYTVTDIKDLTKLGAIYDKTKKYWVYQGKPVMPDQFTFELLDFLHQLTHLSFSKMKALLERSHSPYYMLNRDRTLKNITETCKACAQVNASKSAVKQGTRVRGHRPGTHWEIDFTEIKPGLYGYKYLLVFIDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADPGGGPSSRLTWRVQRSQNPLKIRLTREAP

SEQ ID NO: 14 - protein, 665 aa, Moloney murine leukimia virus
  MISC_FEATURE (1)..(665): env protein
MARSTLSKPLKNKVNPRGPLIPLILLMLRGVSTASPGSSPHQVYNITWEVTNGDRETVWATSGNHPLWTWWPDLTPDLCMLAHHGPSYWGLEYQSPFSSPPGPPCCSGGSSPGCSRDCEEPLTSLTPRCNTAWNRLKLDQTTHKSNEGFYVCPGPHRPRESKSCGGPDSFYCAYWGCETTGRAYWKPSSSWDFITVNNNLTSDQAVQVCKDNKWCNPLVIRFTDAGRRVTSWTTGHYWGLRLYVSGQDPGLTFGIRLRYQNLGPRVPIGPNPVLADQQPLSKPKPVKSPSVTKPPSGTPLSPTQLPPAGTENRLLNLVDGAYQALNLTSPDKTQECWLCLVAGPPYYEGVAVLGTYSNHTSAPANCSVASQHKLTLSEVTGQGLCIGAVPKTHQALCNTTQTSSRGSYYLVAPTGTMWACSTGLTPCISTTILNLTTDYCVLVELWPRVTYHSPSYVYGLFERSNRHKREPVSLTLALLLGGLTMGGIAAGIGTGTTALMATQQFQQLQAAVQDDLREVEKSISNLEKSLTSLSEVVLQNRRGLDLLFLKEGGLCAALKEECCFYADHTGLVRDSMAKLRERLNQRQKLFESTQGWFEGLFNRSPWFTTLISTIMGPLIVLLMILLFGPCILNRLVQFVKDRISVVQALVLTQQYHQLKPIEYEP

SEQ ID NO: 15 - DNA, 9709 bp, Human immunodeficiency virus type 1
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggctgcacgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 16 - protein, 500 aa, Human immunodeficiency virus type 1
  MISC_FEATURE (1)..(500): gag polyprotein
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 17 - protein, 1003 aa, Human immunodeficiency virus type 1
  MISC_FEATURE (1)..(1003): pol polyprotein
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVGQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDGLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 18 - protein, 192 aa, Human immunodeficiency virus type 1
  MISC_FEATURE (1)..(192): viv protein
MENRWQVMIVWQVDRMRINTWKRLVKHHMYISRKAKDWFYRHHYESTNPKISSEVHIPLGDAKLVITTYWGLHTGERDWHLGQGVSIEWRKKRYSTQVDPDLADQLIHLHYFDCFSESAIRNTILGRIVSPRCEYQAGHNKVGSLQYLALAALIKPKQIKPPLPSVRKLTEDRWNKPQKTKGHRGSHTMNGH

SEQ ID NO: 19 - protein, 96 aa, Human immunodeficiency virus type 1
  MISC_FEATURE (1)..(96): vpr protein
MEQAPEDQGPQREPYNEWTLELLEELKSEAVRHFPRIWLHNLGQHIYETYGDTWAGVEAIIRILQQLLFIHFRIGCRHSRIGVTRQRRARNGASRS

SEQ ID NO: 20 - protein, 86 aa, Human immunodeficiency virus type 1
  MISC_FEATURE (1)..(86): tat protein
MEPVDPRLEPWKHPGSQPKTACTNCYCKKCCFHCQVCFMTKALGISYGRKKRRQRRRAHQNSQTHQASLSKQPTSQSRGDPTGPKE

SEQ ID NO: 21 - protein, 116 aa, Human immunodeficiency virus type 1
  MISC_FEATURE (1)..(116): rev protein
MAGRSGDSDEELIRTVRLIKLLYQSNPPPNPEGTRQARRNRRRRWRERQRQIHSISERILSTYLGRSAEPVPLQLPPLERLTLDCNEDCGTSGTQGVGSPQILVESPTVLESGTKE

SEQ ID NO: 22 - protein, 81 aa, Human immunodeficiency virus type 1
  MISC_FEATURE (1)..(81): vpu protein
MQPIIVAIVALVVAIIIAIVVWSIVIIEYRKILRQRKIDRLIDRLIERAEDSGNESEGEVSALVEMGVEMGHHAPWDIDDL

SEQ ID NO: 23 - protein, 854 aa, Human immunodeficiency virus type 1
  MISC_FEATURE (1)..(854): envelope protein
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGCTSMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 24 - protein, 206 aa, Human immunodeficiency virus type 1
  MISC_FEATURE (1)..(206): nef protein
MGGKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 25 - DNA, 1497 bp, Lymphocytic choriomeningitis virus
  exon (1)..(1497): LCMV WE-HPI, GP variant (open reading frame)
  misc_feature (281)..(1379): mutations of <400> 1(2) at pos. 281(94), 329(110), 385(129), 397(133), 463 (155), 521(174), 543(181), 631(211), 793(265), 1039347), 1363(455),
atgggtcagattgtgacaatgtttgaggctttgcctcacatcattgatgaggtcatcaacattgtcattattgtgctcattataatcacgagcatcaaagctgtgtacaatttcgccacctgtgggatattagcactggtcagcttcctttttttggctggtaggtcctgtggcatgtacggccttaatggtcccgacatctataaaggggtttaccagttcaaatcagtggagtttgatatgtctcacttaaatctgacgatgcccaatgcgtgctcagccaacaactctcatcactacatcagtatgggaagctctggactggagctaactttcactaacgactccatccttaatcacaatttttgcaacttaacctccgctttcaacaaaaagacttttgaccatacactcatgagtatagtctcgagtctgcacctcagtattagagggaattccaaccacaaagcagtgtcttgtgattttaacaatggcatcaccattcaatacaacttgtcattttcggacccacagagcgctataagccagtgtaggactttcagaggtagagtcttggacatgtttagaactgcctttggaggaaaatacatgagaagtggctggggctgggcaggttcagatggcaagaccacttggtgcagccaaacaagctatcagtacctaatcatacaaaacaggacttgggaaaaccactgtagatatgcaggcccttttgggatgtctagaatcctctttgctcaggaaaagacaaagtttctcactaggagacttgcaggcacattcacctggaccctgtcagactcctcaggagtagaaaatccaggtggttattgcctgaccaaatggatgatccttgctgcagagctcaaatgttttgggaatacagctgttgcaaaatgtaatgtcaatcatgatgaagagttctgtgacatgctacgactaattgattacaacaaggccgccctgagtaagttcaagcaagatgtagagtctgccttgcatgtattcaaaacaacagtaaattctctgatttccgatcagctgttgatgaggaatcatctaagagatctaatgggggtaccatactgtaattactcaaagttctggtatctggaacatgctaagactggtgagactagtgtacccaagtgctggcttgtcactaatggctcctacttgaatgagacccactttagtgatcaaatcgaacaagaagcagataacatgatcacagagatgttgaggaaggactacataaaaagacaagggagtactcctttagccttaatggatcttttgatgttttcaacatcagcatatctaatcagcatctttctgcatcttgtgaagataccaacacatagacacataaagggcggttcatgtccaaagccacaccgcttgaccaacaaggggatctgtagttgtggtgcattcaaggtgcctggtgtaaaaactatctggaaaagacgctga

SEQ ID NO: 26 - protein, 498 aa, Lymphocytic choriomeningitis virus
  MISC_FEATURE (1)..(498): LCMV variant WE-HPI, glycoprotein
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 27 - DNA, 1497 bp, Artificial Sequence
  LCMV WE-HPI opt: codon-optimized sequence of the glycoprotein
atgggccagatcgtgaccatgttcgaggccctgccccacatcatcgacgaggtgatcaacatcgtgatcatcgtgctcatcatcatcaccagcatcaaggccgtgtacaacttcgccacctgcggcatcctggccctggtgagcttcctgttcctggccggcagaagctgcggcatgtacggcctgaatggccccgatatctacaagggcgtgtaccagttcaagagcgtggagttcgacatgagccacctgaacctcaccatgcccaacgcctgcagcgccaacaatagccaccactacatcagcatgggcagcagcggcctggagttgaccttcaccaacgacagcatcctgaaccacaacttctgcaacctcaccagcgccttcaacaagaaaaccttcgaccacaccctcatgagcatcgtgagcagcctgcacctgagcatcagaggcaacagcaaccacaaggccgtgagctgcgacttcaacaacggcatcaccatccagtacaacctgagcttcagcgatcctcagagcgccatcagccagtgcagaaccttcagaggcagagtgctggacatgttcagaaccgccttcggcggcaagtacatgagaagcggctggggctgggccggcagcgacggcaagaccacctggtgcagccagaccagctaccagtacctcatcatccagaacagaacctgggagaaccactgcagatacgccggacctttcggcatgagcagaatcctgttcgcccaggagaaaaccaagttcctcaccaggagactggccggcaccttcacctggaccctgagcgacagcagcggcgtggagaaccctggcggctactgcctcaccaagtggatgatcctggccgccgagctgaagtgcttcggcaacaccgccgtggccaagtgcaacgtgaaccacgacgaggagttctgcgacatgctgagactcatcgactacaacaaggccgccctgagcaagttcaagcaggacgtggagagcgccctgcacgtgttcaagaccaccgtgaacagcctcatcagcgaccagctgctcatgagaaaccacctgagagacctcatgggcgtgccctactgcaactacagcaagttctggtatctggagcacgccaagaccggcgagaccagcgtgcccaagtgctggctggtgaccaatggcagctacctgaacgagacccacttcagcgaccagatcgagcaggaagccgacaacatgatcaccgagatgctgaggaaggactacatcaagagacagggcagcacccccctggccctcatggatctgctcatgttcagcaccagcgcctacctcatcagcatcttcctgcacctggtgaagatccccacccacagacacatcaagggcggcagctgccccaagccccacagactcaccaacaagggcatctgcagctgcggcgccttcaaggtgcccggcgtgaaaaccatctggaagaggagataa

SEQ ID NO: 28 - DNA, 1476 bp, Lassa virus Variant Josia
  misc_feature (1)..(1476): Lassa virus Variant Josia, Glycoprotein
atgggacaaatagtgacattcttccaggaagtgcctcatgtaatagaagaggtgatgaacattgttctcattgcactgtctgtactagcagtgctgaaaggtctgtacaattttgcaacgtgtggccttgttggtttggtcactttcctcctgttgtgtggtaggtcttgcacaaccagtctttataaaggggtttatgagcttcagactctggaactaaacatggagacactcaatatgaccatgcctctctcctgcacaaagaacaacagtcatcattatataatggtgggcaatgagacaggactagaactgaccttgaccaacacgagcattattaatcacaaattttgcaatctgtctgatgcccacaaaaagaacctctatgaccacgctcttatgagcataatctcaactttccacttgtccatccccaacttcaatcagtatgaggcaatgagctgcgattttaatgggggaaagattagtgtgcagtacaacctgagtcacagctatgctggggatgcagccaaccattgtggtactgttgcaaatggtgtgttacagacttttatgaggatggcttggggtgggagctacattgctcttgactcaggccgtggcaactgggactgtattatgactagttatcaatatctgataatccaaaatacaacctgggaagatcactgccaattctcgagaccatctcccatcggttatctcgggctcctctcacaaaggactagagatatttatattagtagaagattgctaggcacattcacatggacactgtcagattctgaaggtaaagacacaccagggggatattgtctgaccaggtggatgctaattgaggctgaactaaaatgcttcgggaacacagctgtggcaaaatgtaatgagaagcatgatgaggaattttgtgacatgctgaggctgtttgacttcaacaaacaagccattcaaaggttgaaagctgaagcacaaatgagcattcagttgatcaacaaagcagtaaatgctttgataaatgaccaacttataatgaagaaccatctacgggacatcatgggaattccatactgtaattacagcaagtattggtacctcaaccacacaactactgggagaacatcactgcccaaatgttggcttgtatcaaatggttcatacttgaacgagacccacttttctgatgatattgaacaacaagctgacaatatgatcactgagatgttacagaaggagtatatggagaggcaggggaagacaccattgggtctagttgacctctttgtgttcagtacaagtttctatcttattagcatcttccttcacctagtcaaaataccaactcataggcatattgtaggcaagtcgtgtcccaaacctcacagattgaatcatatgggcatttgttcctgtggactctacaaacagcctggtgtgcctgtgaaatggaagagatga